(19) United States
(12) Patent Application Publication
(10) Pub. No.: US 2006/0165715 A1
(43) Pub. Date: Jul. 27, 2006

(54) Gp41 inhibitor

(76) Inventors: Marius G Clore, Bethesda, MD (US); Carole A Bewley-Clore, Bethesda, MD (US); John L Medabalimi, Olney, MD (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/499,094

(22) PCT Filed: Dec. 17, 2002
(86) PCT No.: PCT/US02/40684

§ 371 Date: Oct. 18, 2004

Related U.S. Application Data
(60) Provisional application No. 60/339,751, filed on Dec. 17, 2001.

(51) Int. Cl. A61K 39/21; C12Q 1/70; C07K 14/16

(52) U.S. Cl. 424/188.1; 530/350; 435/5

(57) ABSTRACT

This invention provides a trimeric protein complex that presents an exposed N-terminal coiled-coil domain from the HIV gp41 protein. The preferred embodiment of the invention inhibits membrane fusion mediated by HIV virus. The invention also provides methods to use the trimeric protein complexes as a vaccines to prevent infection by the HIV virus.

### CROSS-REFERENCES TO RELATED APPLICATIONS

This application claims the benefit of provisional U.S. application Ser. No.60/339,751, filed Dec. 17, 2001, which is herein incorporated by reference for all purposes.

### BACKGROUND OF THE INVENTION

Infection by and dissemination of the human immunodeficiency virus (HIV) necessitates virus-cell or cell-cell fusion mediated by envelope (Env) glycoproteins. HIV-1 Env consists of two non-covalently attached proteins, gp120 and gp41, derived by proteolytic cleavage-of gp160 (Freed el al., J. Biol. Chem., 270:23883-23886 (1995)). The molecular events leading to fusion include initial binding of gp120 to CD4, a cellular receptor for HIV Env. The binding triggers conformational changes in gp120 that permit subsequent interactions with the chemokine receptors, the cellular co-receptors CXCR4 or CCR5 (Moore et al., Cur. Opin. Immunol., 9:551-562 (1997)). This results in a further series of conformational changes in the gp120/gp41 oligomer that lead to insertion of the fusion peptide of gp41 into the target membrane and ultimately, membrane fusion.

Both gp120 and gp41 offer potential targets for the inhibition of viral entry, either through drugs or neutralizing antibodies. Neutralizing antibodies directed at gp120 have been difficult to elicit (McMichael et al., Nature Medicine, 5:612-614 (1999)) since the surface of gp120 is heavily glycosylated (Kwong et al., Nature, 393:648-659 (1998)), thereby preventing access to the conserved regions of the molecule that bind the requisite cellular receptors. One very potent inhibitor of fusion directed against gp120 has been discovered: namely, the small protein cyanovirin-N (Boyd et al., Antimicrob. Agents Chemother., 41:1521-1530 (1997)) whose selectivity has been shown to arise as a consequence of specific nanomolar binding to MangGlcNac₂ and the D1D3 isomer of Man₈GlcNac₂ present in abundance on the surface of gp120 (Bewley et al., J. Am. Chem. Soc., 123:3892-3902 (2001)). A potentially more amenable target is afforded by gp41, particularly in the so-called pre-hairpin intermediate state (Chan et al., Cell, 93:681-684 (1998)).

The solution structure of the complete ectodomain of SIV gp41 (Caffrey et al., EMBOJ., 17:4572-4584 (1998)) and crystal structures of various fragments of the ectodomain cores of HIV (Chan et al., Cell, 89:263-273 (1997); Weissenhom et al., Nature, 387:426-430 (1997); Tan et al., Proc. Natl. Acad. Sci. U.S.A., 94:12303-12308 (1997)) and SIV (Malashkevich et al., Proc. Natl. Acad. Sci. U.S.A., 95:9134-9139 (1998)) gp41 have been determined. Each gp41 monomer consists of two long helices at the N- and C-termini connected by a long linker. The core of gp41 is a trimer of hairpins making up a six-helix bundle: three N-terminal helices form a central parallel coiled-coil around which are packed the C-terminal helices in an antiparallel manner. This structure is thought to represent the fusogenic state of gp41 which serves to bring the viral and cell membranes into close proximity, thereby promoting membrane fusion (Chan et al., Cell, 93:681-684 (1998)).

Peptides derived from the C- and N-helices of gp41 inhibit fusion (Wild et al., Proc. Natl. Acad. Sci. U.S.A., 89:10537-10541 (1992); Wild et al., Proc. Natl. Acad. Sci. U.S.A., 91:9770-9774 (1994)). The C-peptides, which are monomeric in solution (Lu et al., Nature Struct. Biol., 2:1075-1082 (1995)), have nanomolar IC₅₀'s (Wild et al., Proc. Natl. Acad. Sci. U.S.A., 91:9770-9774 (1994); Chan et al., Proc. Natl. Acad. Sci. U.S.A., 95:15613-15617 (1998)) and some of these are currently in clinical trials (Kilby et al., Nature Medicine, 4:1302-1307 (1998)). The activity of the N-peptides, on the other hand, is about three orders of magnitude lower (Wild et al., Proc. Natl. Acad. Sci. U.S.A., 89:10537-10541 (1992)), presumably due to aggregation and their inability to form a trimeric coiled-coil in the absence of C-peptide (Lu et al., Nature Struct. Biol., 2:1075-1082 (1995)).

Both the N- and C-peptides are thought to target the pre-hairpin fusion intermediate which persists for many minutes (Chan et al., Cell, 93:681-684 (1998); Furuta et al., Nature Struct. Biol., 5:276-279 (1998)). Formation of the pre-hairpin intermediate in which the N-terminal fusion peptide of gp41 is inserted into the target membrane is postulated to expose the trimeric coiled-coil of N-helices to which the C-peptides bind with high affinity, thereby preventing formation of the fusogenic trimer of hairpins (Chan et al., Cell, 93:681-684 (1998); Furuta et al., Nature Struct. Biol., 5:276-279 (1998)). The N-helix of gp41 has also been targeted by cyclic D-peptide inhibitors derived from phage display (Eckert et al., Cell, 99:103-115 (1999)) and a variety of non-natural binding elements generated by combinatorial chemistry and linked to truncated C-peptides (Ferrer et al., Nature Struct. Biol., 6:953-960 (1999)). The N-peptides are thought to either hinder the formation of the trimeric coiled-coil of N-helices (Weng et al., J. Virol., 72:9676-9682 (1998)) or bind to the C-terminal region of the gp41 ectodomain corresponding to the C-helix in the fusogenic state of gp41 (Chan et al., Cell, 93:681-684 (1998)).

Peptides that target the N-helix are active in the nanomolar range. While the C-helix of gp41 is also a viable target for therapeutic intervention, to date the reported inhibitory activities for peptides that target the C-helix, e.g. N-peptides, are only in the micromolar range. In addition, the N-helices of gp41 could potentially be used as an HIV vaccine or to generate antibodies which could then be used to block fusion between HIV virion or infected cells and uninfected cells. However, in the native protein, the N-helices are shielded by the C-terminal helices, and thus are not exposed in solution. The present invention addresses these and other needs.

### BRIEF SUMMARY OF THE INVENTION

This invention provides compositions for presenting an exposed N-terrninal trimeric coiled-coil domain from the HIV gp41 protein. In addition the invention provides methods to use the exposed N-terminal trimeric coiled-coil domain as a vaccine against infection by HIV, as therapeutic treatment of HIV, and to generate antibodies directed against the exposed N-terminal trimeric coiled-coil domain.

In one aspect the exposed N-terminal trimeric coiled-coil domain is part of a trimeric polypeptide complex consisting of three polypeptide subunits, where each subunit comprises between 30 and 50 amino acids from an N-terminal domain of gp41 protein from HIV at the N-terminus of the subunit, with the proviso that the subunit does not include a carboxy-terminal domain of gp41 protein from HIV. The N-terminal domain of the subunit also has at least 80% sequence identity to an N34_{CCG} protein of FIG. 6b, and has an amino terminus and a carboxy terminus. The N-terminal domain of the subunit further has at least two cysteine residues in the ten residues from the carboxy terminus of the domain, and the cysteine residues are able to cross-link with cysteine residues in two other polypeptide subunits of the trimeric polypeptide complex when the subunits are properly folded. When allowed to assemble with two other subunits into a disulfide bridge stabilized trimeric polypeptide complex, the N-terminal domain of the subunit forms an exposed trimeric coiled-coil domain having at least 40% alpha helical content. In addition, the trimeric polypeptide complex inhibits, cell fusion in an HIV based membrane fusion assay.

In one embodiment, the polypeptide subunits of the trimeric polypeptide include the amino acid sequence of the N34_{CCG} protein of FIG. 6b.

In another aspect the polypeptide subunits of the trimeric polypeptide complex include a second N-terminal domain of gp41 attached to the carboxy terminus of the subunit. These polypeptide subunits can include 1-13 residues of an N-terminal domain of gp41 or have at least 80% identity to an N35_{CCG}-N13 protein of FIG. 6b.

In another embodiment, the N-terminal domain of the subunit forms an exposed trimeric coiled-coil domain with at least 50% alpha helical content when allowed to assemble with two other subunits into a disulfide bridge stabilized trimeric polypeptide complex. In a further embodiment, the polypeptide subunits further comprise a His-tag sequence.

In a further aspect, the trimeric polypeptide complex is included in a pharmaceutical excipient suitable for administration to a human, in an amount sufficient to generate an immune response.

In one aspect the invention provides a method of protecting a human from HIV infection by administering to the human an amount of an immunogenic composition comprising an exposed N-terminal trimeric coiled-coil domain from the HIV gp41 protein. In one aspect the exposed N-terminal trimeric coiled-coil domain is part of a trimeric polypeptide complex consisting of three polypeptide subunits, where each subunit comprises between 30 and 50 amino acids from an N-terminal domain of gp41 protein from HIV at the N-terminus of the subunit, with the proviso that the subunit does not include a carboxy-terminal domain of gp41 protein from HIV. The N-terminal domain of the subunit also has at least 80% sequence identity to an N34_{CCG} protein of FIG. 6b, and has an amino terminus and a carboxy terminus. The N-terminal domain of the subunit further has at least two cysteine residues in the ten residues from the carboxy terminus of the domain, and the cysteine residues are able to cross-link with cysteine residues in two other polypeptide subunits of the trimeric polypeptide complex when the subunits are properly folded. When allowed to assemble with two other subunits into a disulfide bridge stabilized trimeric polypeptide complex, the N-terminal domain of the subunit forms an exposed trimeric coiled-coil domain having at least 40% alpha helical content. In addition, the trimeric polypeptide complex inhibits cell fusion in an HIV based membrane fusion assay.

In one embodiment, the polypeptide subunits of the trimeric polypeptide used as an immunogen include the amino acid sequence of the N34_{CCG} protein of FIG. 6b.

In another aspect the polypeptide subunits of the trimeric polypeptide complex used as an immunogen include a second N-terminal domain of gp41 attached to the carboxy terminus of the subunit. These polypeptide subunits can include 1-13 residues of an N-terminal domain of gp41 or have at least 80% identity to an N35_{CCG}-N13 protein of FIG. 6b.

In another embodiment, the N-terminal domain of the subunit forms an exposed trimeric coiled-coil domain with at least 50% alpha helical content when allowed to assemble with two other subunits into a disulfide bridge stabilized trimeric polypeptide complex. In a further embodiment, the polypeptide subunits used as an inmmunogen fuirther comprise a His-tag sequence.

The present invention also provides an immunogen capable of inducing a response against an exposed trimeric coiled-coil domain from an N-terminal domain of gp41 protein from HIV comprising a trimeric polypeptide complex with an exposed N-terminal trimeric coiled-coil domain from the HIV gp41 protein where the trimeric polypeptide complex is soluble in an aqueous solution of pH 7 at a concentration of at least 0.5 micromolar. In one aspect the exposed N-terminal trimeric coiled-coil domain is part of a trimeric polypeptide complex consisting of three polypeptide subunits, where each subunit comprises between 30 and 50 amino acids from an N-terminal domain of gp41 protein from HIV at the N-terminus of the subunit. The N-terminal domain of the subunit also has at least 80% sequence identity to an N34_{CCG} protein of FIG. 6b, and has an amino terminus and a carboxy terminus. The N-terminal domain of the subunit further has at least two cysteine residues in the ten residues from the carboxy terminus of the domain, and the cysteine residues are able to cross-link with cysteine residues in two other polypeptide subunits of the trimeric polypeptide complex when the subunits are properly folded. When allowed to assemble with two other subunits into a disulfide bridge stabilized trimeric polypeptide complex, the N-terminal domain of the subunit forms an exposed trimeric coiled-coil domain having at least 40% alpha helical content. In addition, the trimeric polypeptide complex inhibits cell fusion in an HIV based membrane fusion assay.

In one embodiment, the polypeptide subunits of the trimeric polypeptide include the amino acid sequence of the N34_{CCG} protein of FIG. 6b.

In another aspect the polypeptide subunits of the trimeric polypeptide complex include a second N-terminal domain of gp41 attached to the carboxy terminus of the subunit. These polypeptide subunits can include 1-13 residues of an N-terminal domain of gp41 or have at least 80% identity to an N35_{CCG}-N13 protein of FIG. 6b.

In another embodiment, the N-terminal domain of the subunit forms an exposed trimeric coiled-coil domain with at least 50% alpha helical content when allowed to assemble with two other subunits into a disulfide bridge stabilized trimeric polypeptide complex. In a further embodiment, the polypeptide subunits further comprise a His-tag sequence.

In one embodiment, the carboxy terminus of the N-terminal domain of the subunit is fused to an amino terminus of a six helix bundle domain, and further, has at least 90% alpha helical content when the N-terminal domain of the subunit is fused to a six helix bundle domain and allowed to assemble with two other subunits into a disulfide bridge stabilized trimeric protein. In another embodiment, the N-terminal domain of the subunit forms a trimeric protein having 90% alpha helical content when the N-terminal domain of the subunit is fuised to the six helix bundle domain of SEQ ID NO:4 and allowed to assemble with two other subunits into a disulfide bridge stabilized trimeric protein. The six helix bundle domain can be selected from the group consisting of: the gp41 protein of HIV-1, the gp41 protein of SIV, and GCN4.

In a further embodiment, the six helix bundle domain comprises an N34 domain linked to a C28 domain where the N34 domain is has between 30 and 50 amino acid residues having an amino terminus and carboxy terminus of N34, and where at least 30 amino acid residues of the total amino acid residues of N34 have more than an 80% sequence identity to SEQ ID:2, and the C28 domain has between 25 and 45 amino acid residues having an amino terminus and carboxy terminus of C28, and where at least 28 amino acid residues of the total amino acid residues of C28 have more than an 80% sequence identity to SEQ ID:3, and where the carboxy terminus of the N34 domain is linked to the amino terminus of the C28 domain by a linker of between 4 and 12 amino acids. In another embodiment, the N-terminal domain of gp41 protein from HIV comprises SEQ ID NO: 1. In a further embodiment, the polypeptide subunits comprise SEQ ID NO:5. The trimeric polypeptide complex made of subunits that include a six-helix bundle domain can be included in a pharmaceutical excipient suitable for administration to a human, in an amount sufficient to generate an immune response.

In one aspect the invention provides a method of protecting a human from HIV infection by administering to the human an amount of a immunogenic composition comprising a trimeric polypeptide complex consisting of three polypeptide subunits where each subunit comprises between 30 and 50 amino acids from an N-terminal domain of gp41 protein from HIV at the N-terminus of the subunit and a six helix bundle domain. In one embodiment, the polypeptide subunits comprise SEQ ID NO:5. In another embodiment, the composition is administered parenterally.

The present invention also provides an immunogen capable of inducing a response against an exposed trimeric coiled-coil domain from an N-terminal domain of gp41 protein from HIV comprising a trimeric polypeptide complex with an exposed N-terminal trimeric coiled-coil domain from the HIV gp41 protein and a six helix bundle where the trimeric polypeptide complex is soluble in an aqueous solution of pH 7 at a concentration of at least 0.5 micromolar.

### DEFINITIONS

A “trimeric polypeptide complex” is a protein complex consisting of three polypeptide subunits. In some embodiments, the trimeric polypeptide complex consists of three identical polypeptide subunits and is thus a “homotrimeric polypeptide complex”. A “polypeptide subunit” is a single amino acid chain or monomer that in combination with two other polypeptide subunits forms a trimeric polypeptide complex. For convenience, the portion of the subunit that is able to formn an exposed trimeric coiled-coil domain when properly folded is referred to as an “N-terminal domain of a subunit.” Thus, the N-terminal domain of a subunit is a version of the N-terminal domain of gp41 protein from HIV described below. In some embodiments additional protein domains are added to the carboxy-terminus of the N-terminal domain of a subunit. For example, an additional N-terminal domain of gp41 protein from HIV or portions of such domains can be added, e.g., an N13 domain. In some embodiments a six helix bundle is added to the N-terminus of the subunit. Exemplary six helix bundle domains include domains from gp41 protein from HIV, gp41 protein from SIV, and GCN4. The six helix bundle domains are frequently engineered to suit the needs of the user.

An “N-terminal domain of gp41 protein from HIV” is a portion of the gp41 protein (the transmembrane subunit of HIV envelope) from the N-terminus that forms trimeric coiled-coil structures when properly folded. The N-terminal domain of gp41 protein from HIV can include between 30 and 50 amino acid residues and is based on the sequence of the native N-terminal domain of gp41 of HIV. An N-terminal domain of gp41 protein from HIV is frequently engineered to suit the needs of the user. N-terminal domains of gp41 can include N13, N34, N35, and N36 which encompass residues 546-558, 546-579, 546-580, and 546-581, of HIV-1 Env, respectively. N34_{CCG} and N35_{CCG} correspond to N34 and N35, respectively, with Leu576, Gln577 and Ala578 of HIV-1 Env substituted by Cys, Cys and Gly, respectively. N35_{CCG}-N13 is a 48 residue peptide comprising N35_{CCG} immediately followed by N13. N₃₆^{Mut[e,g]} is a peptide derived from N36 which contains 9 substitutions at positions e and f of the helical wheel (defined in the context of the gp41 trimer of hairpin structure) corresponding to residues 549, 551, 556, 558, 563, 565, 570, 572 and 577 of HIV-1 Env. (Bewley et al., J. Biol. Chem. 277:14238-14245 (2002)).

A “carboxy-terminal domain of gp41 protein from HIV” is a portion of the gp41 protein (the transmembrane subunit of HIV envelope) from the carboxy-terminus that form external helices that pack around the internal trimeric coiled-coil domain of the native protein. Carboxy-terminal domains of gp41 can include C28 and C34 which encompass residues 628-655 and 628-661 of HIV-1 Env, respectively.

N_{CCG}-gp41 is a chimeric protein comprising an N-terminal domain of gp41 protein from HIV and a carboxy-terminal domain from gp41 from HIV. N_{CCG}-gp41 consists of N35_{CCG} fused onto the minimal thermostable ectodomain core of gp41: N35_{CCG}-N34-(L6)-C28, where L6 is a six residue linker (SGGRGG).

Alpha helical content refers to a repeating protein structure formed because of conformational constraints on the protein. Formation of the rod-like a-helix is determined by the primary amino acid sequence of the protein. While the polypeptide main chains are tightly coiled and form the inner part of the rod-like structure, the amino acid side chains extend out in a helical array. The structure is stabilized by hydrogen bonding and conformational constraints around the peptide bond. With regard to the exposed trimeric coiled-coil domains of the-present invention, the alpha helical content is preferably at least 40%, but in some embodiments is 45%, 50%, 55%, 60%, 65%, 70%, 75%, 80%, 85%,90%, 95%, or greater than 95%.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, preferably 65%, 70%, 75%, 80%, 85%, 90%, or 95% identity over a specified region such as amino acids 102-514 of SEQ ID NO:3), when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Preferably, the identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the prograrn parameters. For sequence comparison of nucleic acids and proteins to N_{CCG}-gp⁴l nucleic acids and proteins, the BLAST and BLAST 2.0 algorithms and the default parameters discussed below are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermnal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For high stringency hybridization, a positive signal is at least two times background, preferably 10 times background hybridization. Exemplary high stringency or stringent hybridization conditions include: 50% formamide, 5×SSC and 1% SDS incubated at 42° C. or 5×SSC and 1% SDS incubated at 65° C., with a wash in 0.2×SSC and 0.1% SDS at 65° C.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min.

“Immunogen” or “immunogenic” refer to a composition that elicits the production of an antibody that binds a component of the composition when administered to an animal, or that elicits the production of a cell-mediated immune response against a component of the composition.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990))

For preparation of monoclonal or polyclonal antibodies, any technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985)). Techniques for the production of single chain antibodies (U.S. Pat. No. 4,946,778) can be adapted to produce antibodies to polypeptides of this invention. Also, transgenic mice, or other organisms such as other mammnals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

An “anti- N35” antibody is an antibody or antibody fragment that specifically binds a polypeptide encoded by an N35 gene, CDNA, or a subsequence thereof

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyrne, toxin, hormone, growth factor, drug, etc.; or (b) the variable region, or a portion thereof, is altered, replaced or exchanged with a variable region having a different or altered antigen specificity.

The term “immunoassay” is an assay that uses an antibody to specifically bind an antigen. The immunoassay is characterized by the use of specific binding properties of a particular antibody to isolate, target, and/or quantify the antigen.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to N35, as shown in SEQ ID NO:1, or splice variants, or portions thereof, can be selected to obtain only those polyclonal antibodies that are specifically immunoreactive with N35 and related proteins and not with other proteins. This selection may be achieved by subtracting out antibodies that cross-react with other molecules. In addition, polyclonal antibodies raised to N35 polymorphic variants, alleles, orthologs, and conservatively modified variants can be selected to obtain only those antibodies that recognize N35, but not other closely related proteins. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Typically a specific or selective reaction will be at least twice background signal or noise and more typically more than 10to 100 times background.

### BRIEF DESCRIPTION OF THE DRAWINGS

FIG. 1: FIG. 1 illustrates the design of the N_{CCG}-gp41 chimera. (a) Sequence of N_{CCG}-gp41. Residue numbering for N_{CCG}-gp41 starts with 1. The corresponding residue numbering for HIV-1 Env is shown in italics and starts at 546. The location of the residues in a helical wheel is indicated by the small letters in italics (a-g) below the amino acid sequence. The sequence comprises residues 546-580 of HIV-1 Env (residues 1-35 and denoted as N35) with Leu576, Gln577 and Ala578 (residues 31-33 of N_{CCG}-gp41) mutated to Cys, Cys and Gly, respectively; followed by residues 546-579 of Env (residues 36-69 and denoted as N34); a six residue linker (residues 70-75); and finally residues 628-655 of Env (residues 76-103 denoted as C28). (b) Model of N_{CCG}-gp41. The structure of the N34-(L6)-C28 portion of N_{CCG}-gp41 has been solved crystallographically (Tan et al., Proc. Natl. Acad. Sci. U.S.A., 94:12303-12308 (1997)). N35 was grafted onto the N-terminal end of the crystal structure to generate a 69 residue continuous a-helix comprising N35 and N34. The three subunits are depicted and the location of the three intersubunit disulfide bonds is shown in light grey. Detailed side (c) and top (d) views illustrating that the three intersubunit disulfide bonds can readily be formed with good stereochemistry. The backbone is shown as a Cα trace and the disulfide bonds are light grey.

FIG. 2: FIG. 2 illustrates construction of synthetic N-gp41 and N_{CCG}-gp⁴¹ genes for expression in E. coli. The N-gp41 coding sequence and its complementary sequence were synthesized in four fragments of approximately equal size and purified by polyacrylimide gel electrophoresis. 1F, 2F, 3F and 4F denote the four fragments; the complementary fragments are 1R, 2R, 3R and 4R, respectively. Each of the fragments was phosphorylated except for 1F and 4R. Fragments 1F and 1R, 2F and 2R, 3F and 3R, 4F and 4R were annealed and then ligated. The assembled full-length N-gp41 DNA was isolated by gel electrophoresis and subsequently cloned into the Ndel and BamH1 sites of the pET11a vector. The DNA sequence of two individual clones was confirmed by sequencing. The L31C, Q32C and A33G mutations were introduced into the N-gp41 DNA sequence using the Quick-Change mutagenesis protocol (Stratagene, CA) to generate the N_{CCG}-gp41 sequence. The underlined sequence denotes the forward and reverse primers used in which the sequence CTGCAAGCG (bold letters) was changed to TGTTGTGGC to encode C31, C32 and G33 in the N_{CCG}-gp41 protein. The amino acid sequence is shown below the nucleotide sequence. Note that while the amino acid sequences of N34 and the first thirty-four residues of N35 are identical, their nucleotide sequences are different.

FIG. 3: FIG. 3 illustrates the biophysical characterization of N_{CCG}-gp41. (a) Analysis of purified native N_{CCG}-gp41 by size-exclusion column chromatography. N_{CCG}-gp41 was fractionated on a Superdex-200 column in 50 mM sodium formate buffer, pH 3.0 and 0.2 M GnHCl at room temperature. The inset shows the SDS-PAGE analysis of N_{CCG}-gp41: lane 1, purified N_{CCG}-gp41 under non-reducing conditions after reverse-phase HPLC but before protein folding; lane 2, N_{CCG}-gp41 under non-reducing conditions after protein folding by dialysis of the protein from 35% acetonitrile/0.05% trifluoroacetic acid into 50 mM sodium formate buffer, pH 3; lane 3, the same fraction of N_{CCG}-gp41 shown in lane 2 under reducing conditions following treatment with 0.55 M 2-mercaptoethanol. The positions of N_{CCG}-gp41 trimer, dimer and monomer are indicated by the letters T, D and M, respectively, on the left-hand side of the inset; the positions of molecular weight markers in kDa are indicated on the right-hand side of the inset. All samples were heated to 90° C. for two minutes in the presence of 1.5% SDS and 17 μM protein in 50 mM Tris-HCl, pH 8.0, with or without 2-mercaptoethanol, prior to loading on the gel. (b) CD spectrum of N_{CCG}-gp41 (9.94 μM trimer in 10 mM sodium formate buffer, pH 3 at ambient temperature).

FIG. 4: FIG. 4 illustrates the inhibition of HIV-1 envelope-mediated cell fusion by the N_{CCG}-gp41 chimera and the gp41 derived peptides C34 and N36. The C34 peptide corresponds to residues 628-631 on HIV-1 Env, the C-helix of gp41; the N36 peptide corresponds to residues 546-581 of Env, the N-helix of gp41. Solid circles, N_{CCG}-gp41; open circles, C34; open squares, N36. Vertical bars indicate standard. The solid lines represent best-fits to the data using the simple activity relationship: % fusion=100/(1+[I]/IC50) where [I] is the inhibitor concentration. The IC50's for N_{CCG}-gp41, C34 and N36 are 16.1±2.8 nM, 2.3±0.5 nM and 16.4±1.8 μM, respectively.

FIG. 5. FIG. 5 depict inhibition of HIV-1 Env-mediated cell fusion by targeting the pre-hairpin intermediate state of gp41. In the pre-hairpin intermediate state, formed subsequent to the interaction of gp120 with CD4 and the chemokine coreceptor, the trimeric coiled-coil of N-helices as the C-terminal region of the gp41 ectodomain are exposed. The pre-hairpin intermediate subsequently collapses to form a trimer of hairpins (whose structure has been solved by NMR and crystallographically) bringing the target and viral membranes into aposition. There are three classes of inhibitors that target the pre-hairpin intermediate and prevent its collapse into the trimer of hapins, thereby rendering it fusion incompetent. Class 1 (e.g. C34) target the exposed trimeric coiled-coil of N-helices; class 2 (e.g. N_{CCG}-gp41, N34_{CCG} and N35_{CCG}-N13) target the C-region; and class 3 (e.g. N36^{Mut(e,g)} interact with the pre-hairpin state to form heterotrimers.

FIG. 6. FIG. 6 depicts design of N34_{CCG} and N35_{CCG}-N13. a, Models of N_{CCG}-gp41 ( ), N35_{CCG}-N13 and N34_{CCG}. The N34-(L6)-C28 gp41 core has been solved crystallographically. The model of N_{CCG}-gp41 was constructed by grafting N35 onto the N-terminus of the crystal structure to generate a contiguous 69 residue helix comprising N35 and N34. The three intermolecular disulfide bridges formed by the two cysteines introduced at positions 576 and 577 are shown at the carboxy-terminus, and the three subunits of the timer are shown black grey and light grey. The models of N35_{CCG}-N13 and N34_{CCG} are directly derived from N_{CCG}-gp41. b, Sequences of N35_{CCG}-N13, N34_{CCG} and N36^{Mut(e,g)}. The residue numbering is that of HIV-1 Env; the engineered Cys-Cys-Gly at positions 576-578 which have replaced the wild type Leu-Gln-Ala sequence are shown. N13 refers to residues 546-558 of HIV-1 env. The mutations in the native sequence of N36 that were introduced into N36^{Mut(e,g)} are shown in light grey. The letters a-g indicate the positions in a helical wheel presentation.

FIG. 7. FIG. 7 depicts biochemical characterization of N34_{CCG} and N35_{CCG}-N13. a, Analysis of purified and folded N_{CCG}-gp41 (black line), N34_{CCG} plus C34 peptide complex (dashed line), and N34_{CCG} (grey line) by size-exclusion column chromatography. The proteins were fractionated on a Superdex-200 column in 50 mM sodium formate buffer, pH 3.0 and 0.2 M GnHCl at room temperature. b, SDS-PAGE analysis of Ncco-gp41 (lanes 1 and 2), N34_{CCG} (lanes 3 and 4) and N35_{CCG}-N13 (lanes 5 and 6). All samples were heated to 90° C. for 2 min. in the presence of 1.5% SDS in 50 mM Tris-HCl, pH 8.0, without (− lanes) or with (+ lanes) β-mercaptoethanol (β-ME), prior to loading on the gel. T1, D1, M1 indicate the positions of the trimer, dimer and monomer, respectively of N_{CCG}-gp41; T2 and M2 indicate the positions of the trimer and monomer, respectively, of N34_{CCG}; and T3 and M3 the positions of the trimer and monomer, respectively of N35_{CCG}-N13.

FIG. 8. FIG. 8 depicts characterization of disulfide-linked trimeric N34_{CCG} and N35_{CCG}-N13 by CD spectroscopy. CD spectra of the trimeric forms of N34_{CCG} (10 μM) and N35_{CCG}-N13 (8 μM) were recorded in 20 mM sodium formate buffer, pH 3 at ambient temperature.

FIG. 9. FIG. 9 depicts inhibition of HIV-1 Env-mediated cell fusion by N34_{CCG}, N35_{CCG}-N13 and N_{CCG}-gp41. Open circles, N34_{CCG}; Solid circles, N35_{CCG}-N13; Diamonds, N_{CCG}-gp41. The solid lines represent best fits to the data using the simple activity relationship: % fusion=100/(1+[I]/IC₅₀) where I is the inhibitor concentration. The calculated IC₅₀ values for N34_{CCG}, N35_{CCG}-N13 and N_{CCG}-gp41 are 96±7 nM, 15.5±1.3 nM and 19.3±1.4 nM, respectively.

### DETAILED DESCRIPTION OF THE INVENTION

I. Introduction

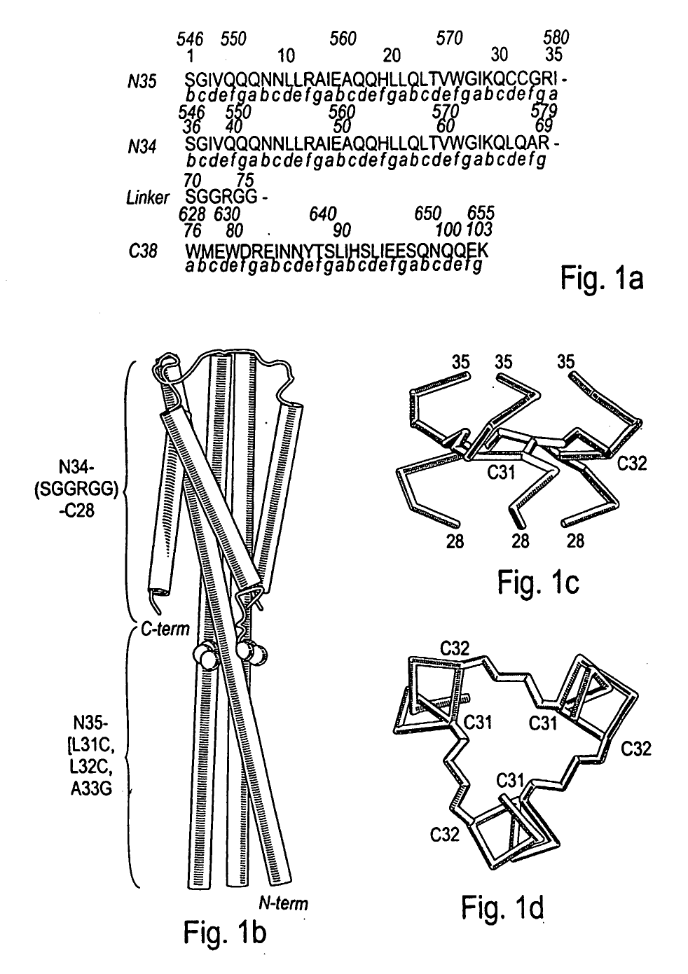

The present invention provides a novel, trimeric polypeptide complexes that expose the N-terminal trimeric coiled coil domain of HIV gp41 protein. One such molecule is the chimeric gp41 molecule, N_{CCG}-gp41 (FIG. 1), in which the N-helix of HIV gp41 is grafted in helical phase onto the N-terminus of a minimal thermostable trimeric core (six-helix bundle) of gp41 and stabilized by intermolecular disulfide bridges. (Louis et al., J Biol Chem. 276:29485-9 (2001)). Using conventional molecular biology and protein purification methods, gp41 is isolated in the fusogenic state, not the pre-hairpin intermediate. That is, the N-terminal coiled-coil is masked by the C-terminal α-helices. Attempts to expose the N-terminal trimeric coiled-coil by expressing the N-terminal peptides failed because the peptides aggregated and did not formn a trimeric coiled-coil structure on their own. The present invention, N_{CCG}-gp41, is a trimeric polypeptide complex engineered to present a stable and exposed trimeric coiled-coil of N-helices from gp41. N_{CCG}-gp41 inhibits membrane fusion at nanomolar concentrations, presumably by binding to the C-terminal helices of gp41 in the pre-hairpin intermediate. In addition to its fusion inhibitory properties, N_{CCG}-gp41 presents conformational epitopes suitable for use as a vaccine or for the generation of fusion inhibitory antibodies directed against the exposed N-helices of gp41 in the pre-hairpin intermediate state. Embodiments of N_{CCG}-gp41 with enhanced solubility at neutral pH are also described.

Other trimeric polypeptide complexes that expose the N-terminal trimeric coiled coil of HIV gp41 protein are also described.

II. Design of the Trimeric Polypeptide Complex

The design of the trimeric polypeptide complex takes advantage of the molecular and structural features of fusion proteins from retroviruses. Briefly, the fusion proteins comprise an exposed N-terminal trimeric coiled-coil domain from HIV gp41 protein. In some embodiments, the trimeric polypeptides also comprise an internal trimeric coiled-coil domain, a linker domain and C-terminal helices that pack around the internal trimeric coiled-coil domain. The six-helix bundle, formed by the internal coiled-coil domain, the linker domain, and the external C-terminal domains, can serve as a scaffold for a stable exposed trimeric coiled-coil domain. Other domains and modifications for stabilizing the exposed N-terminal trimeric coiled-coil domain from HIV gp41 protein are also described.

The three-dimensional structures of the protein domains that comprise the present invention are known. (Chan et al., Cell, 89:263-273 (1997); Weissenhorn et al., Nature, 387:426-430 (1997); Tan et al., Proc. Natl. Acad. Sci. U.S.A., 94:12303-12308 (1997); Malashkevich et al., Proc. Natl. Acad. Sci. U.S.A., 95:9134-9139 (1998)) Structural data can distinguish between amino acids that are internal and more likely to contribute to maintenance of structure, and amino acids that are on the surface of a protein and thus able to interact with other molecules and contribute more directly to the function of the protein. Thus, amino acid residues can be categorized, inter alia, as either contributing to the structure of the protein or to its function.

Because amino acid sequence identity requirements are not as stringent for maintenance of structure, different sequence identity requirements can be applied to structural and functional amino acid residues. (Chothia and Lesk, EMBO J. 5:823-826, (1986)). In addition, amino acids that are on the surface, but not critical for function of the protein can be mutated in order to manipulate biophysical characteristics of the protein. For example, mutation of external, charged residues can affect the solubility of the protein at a particular pH.

A. Features of the Domains That Make Up the Trimeric Polypeptide Complex.

The domains that make up the present invention have been well-characterized and their structure is known. (Chan et al., Cell, 89:263-273 (1997); Weissenhorn et al., Nature, 387:426-430 (1997); Tan et al., Proc. Natl. Acad. Sci. U.S.A., 94:12303-12308 (1997); Malashkevich et al., Proc. Natl. Acad. Sci. U.S.A., 95:9134-9139 (1998)).

1. Protein Structures Which Comprise the Trimeric Polypeptide Complex.

In some embodiments, the trimeric polypeptide complex comprises two types of protein structures: α-helices, and trimeric coiled-coils, which are made up of α-helices.

a. α-helix Structure.

An α-helix is a repeating protein structure formed because of conformational constraints on the protein. Formation of the rod-like α-helix is determined by the primary amino acid sequence of the protein. While the polypeptide main chains are tightly coiled and form the inner part of the rod-like structure, the amino acid side chains extend out in a helical array. The structure is stabilized by hydrogen bonding and conformational constraints around the peptide bond. (Stryer, Biochemistry, 3^{rd} ed., 1988.)

b. Coiled-coil Structure.

A coiled-coil is a multimeric protein structure formed by the interaction of multiple α-helices. The α-helical strands of a coiled-coil protein are in helical register, allowing the amino acid side chains of the different strands to interact. The primary amino acid sequence of coiled-coil α-helices is characterized by the presence of heptad repeats. The heptad repeats in the primary sequence correspond to approximately two turns of the α-helix. The amino acid residues of the heptad repeats are denoted as abcdefg and their structure is often represented as a helical wheel. Residues a and d are hydrophobic and interact with residues a and d from one or more other α-helices to form a tight fitting hydrophobic core. Residues b, c, and f are hydrophilic and are found on the outside of the coil where they interact with the solvent and other molecules. Residues at the e and g positions are important for stability and oligomerization of the coiled-coil structure. (Stryer, Biochemistry, 3^{rd} ed., 1988; Kohn et al., J. Mol. Biol. 283:993-1012 (1998))

2. Relationship Between the Amino Acid Sequence of Trimeric Polypeptide Complexes and Their Structure or Function.

A trimeric polypeptide complex can includes structural domains and functional domains. As described below, the structural domain serves as a scaffold and the functional domain is an exposed trimeric coiled-coil structure. The surface amino acids of the exposed trimeric coiled-coil domain contribute to function either by interacting with the pre-hairpin intermediate of gp41 to block fusion or by presenting conformational epitopes used to raise antibodies against the domain. In some embodiments, a single domain has both structural and functional roles.

The tolerance for changes in amino acid sequence of the present invention will depend on whether the amino acids serve a structural role or a functional role. As a rule of thumb, when considering the conserved structural cores of two divergent proteins, as long as amino acid sequence identity between the divergent proteins is greater than 30%, homologous structural features will be preserved. (Chothia and Lesk, EMBO J. 5:823-826, (1986)). This rule is not true for amino acid sequences that contribute to polypeptide functions such as enzymatic activity, binding to specific protein domains, or formation of epitopes that are recognized by antibodies. For these amino acid sequences greater sequence identity, on the order of 80%, will be required to maintain protein function. In order to apply the correct rules of homology, efforts will be made to distinguish between amino acids that contribute primarily to maintenance of structure and amino acid that contribute primarily to protein function.

Amino acid sequences can be mutated to alter the biophysical features of the protein, so long as the structure of the protein fold is maintained. For example, the solubility of the N_{CCG}-gp41 protein varies with pH. To modify this feature of the protein, appropriate amino acids can be mutated to increase solubility at a desired pH.

Additional domains can be added to the core structural and functional domains described above. One of skill in the art will recognize that useful protein domains can be added without interfering with the structure or function of the present invention. For example, well characterized protein tags can be added to aid in protein purification or detection by immunoassays. Protein sequences can also be added to facilitate encapsulation of the protein in liposomes or to adjust the net charge of the protein by adding amino acids of a desired charge.

B. The N-terminal Trimeric Coiled Coil Domain of gp41

The N-terminus of gp41 is an exposed trimeric coiled-coil domain. Residues on the surface of the N-terminal trimeric coiled-coil interact with native gp41 protein, thereby blocking viral infection or cell fusion, and also present conformational epitopes for recognition by antibodies directed against N-terminal trimeric coiled-coil.

1. Stabilization of the Exposed Coiled-coil Domain.

Without intervention, monomers of the N-terminal gp41 domain do not associate to form a stable trimeric coiled-coil structure in solution. Engineering of the N-terminal gp41 domain provides the exposed trimeric coiled-coil N-terminal domain as a stabilized molecule to ensure monomers do not dissociate, even at very low concentrations. Thus, the protein is functional at very low concentrations, e.g. nanomolar concentrations. Two methods can be used to stabilize the trimeric protein complex: disulfide bonds between monomeric subunits, and attachment to a stable helix bundle domain. Once stabilized, the exposed N-terminal trimeric coiled-coil domain from HIV gp41 protein has measurable alpha helical content, as measured by circular dichroism. The alpha helical content is preferably at least 40%, but in some embodiments is 45%, 50%, 55%, 60%, 65%, 70%, 75%, 80%, 85%, 90%, 95%, or greater than 95%.

a. Disulfide Bonds.

Disulfide bonds can conveniently be used to covalently link polypeptides containing cysteine residues. Disulfide bonds are formed by the oxidation of cysteine residues. If appropriate cysteine residues are not present in the polypeptide, the DNA sequence encoding the protein can be mutagenized to provide appropriate cysteine residues.

For stabilization of a trimeric coiled-coil structure with disulfide bonds, each monomeric subunit has at least two cysteine residues to allow covalent bonding to each of the other two monomers. The cysteine residues are then positioned in the α-helix comprising the coiled-coil to allow interaction of the sulfide atoms. For example, using the letter designations for a helical wheel, adjacent d and e positions can be used to allow interaction between cysteine residues on monomers, thereby stabilizing the trimeric complex..

The polypeptide N_{CCG}-gp41 provides an example of a trimeric helical coiled-coil domain that has been stabilized by the addition of disulfide bridges. As can be seen in FIG. 1a, amino acids 31 and 32 are cysteine residues. Those amino acids correspond to positions d and e of the helical wheel. Similar amino acid changes provided stabilizing cysteine residue in the N35_{CCG}-N13 molecule and the N34_{CCG} molecule.

b. Attachment of an Exposed Trimeric Coiled-coil Domain to a Stable Helix Bundle Domain.

The exposed trimeric coiled-coil is further stabilized through attachment to a stable helix bundle. To do so, the chelix of a monomer is attached in helical phase to the internal α-helix of a second helix bundle. The source of the helix bundle domain is not critical, so long as it forms a stable trimeric complex and the α-helices can be attached in helical register. In some embodiments, the helix bundle domain is a six helix bundle domain. In some embodiments, the helix bundle domain is a three helix bundle domain. In alternative embodiments, a second helix bundle is not attached to the exposed trimeric coiled-coil.

2. Relationship of the Amino Acid Sequence of N35 to its Function.

The functional residues of N_{CCG}-gp41, N35_{CCG}-N13, and N34_{CCG} are on the surface of the N-termninal gp41 exposed trimeric coiled-coil domain. Using a helical wheel as a model, the surface residues are at positions b, c,f e, and g. Thus, sequence identity of at least 80% should be maintained for those residues identified as being on the surface of the N-terminal gp41 domain and contributing to the function of the protein. FIG. 1a and Table 1 show the residues of the N-terminal gp41 domain that correspond to helical wheel positions b, c, f, e, and g.

TABLE 1
Surface Residues of the N-terminal gp41 Domain

| Residue Number | Amino Acid | Helical Wheel Position |
|---|---|---|
| 1 | S | b |
| 2 | G | c |
| 4 | V | e |
| 5 | Q | f |
| 6 | Q | g |
| 8 | N | b |
| 9 | N | c |
| 11 | L | e |
| 12 | R | f |
| 13 | A | g |
| 15 | E | b |
| 16 | A | c |
| 18 | Q | e |
| 19 | H | f |
| 20 | L | g |
| 22 | Q | b |
| 23 | L | c |
| 25 | V | e |
| 26 | W | f |
| 27 | G | g |
| 29 | K | b |
| 30 | Q | c |
| 32 | C | e |
| 33 | G | f |
| 34 | R | g |

The internal amino acids of the exposed trimeric coiled-coil domain maintain the structure of the domain and are less critical for the function of the protein. Thus, amino acid residues at positions a and d can be altered more extensively. FIG. 1a and Table 2 show the residues of the N-terminal gp41 domain that correspond helical wheel positions a and d. Sequences with identity of 40% at those positions should be able to maintain the required structure of the protein.

TABLE 2
Internal Amino Acids of the N-terminal gp41 Domain

| Residue Number | Amino Acid | Helical Wheel Position |
|---|---|---|
| 3 | I | d |
| 7 | Q | a |
| 10 | L | d |
| 14 | I | a |
| 17 | Q | d |
| 21 | L | a |
| 24 | T | d |
| 28 | I | a |
| 31 | C | d |
| 35 | I | a |

C. The Helix Bundle Domain.

1. Structure of a Six Helix Bundle Domain.

The structure of a six helix bundle domain consists of an internal α-helical domain joined to a linker, which is in turn, joined to external helical domains. The internal α-helices forms trimeric coiled-coil structures. Hydrophobic interactions between the a and d amino acids are on the interior of the coil. The external helices pack around the internal trimeric coiled-coil domain in an antiparallel fashion.

N34-L6-C28, the minimal trimeric core of the ectodomain of HIV gp41, is an exemplary six helix bundle protein and the structure of the protein has been solved. The amino acids at positions a, d, e, and g in the internal trimeric coiled-coil or N34, are of similar hydrophobicity. (Tan et al., Proc. Natl. Acad. Sci. U.S.A., 94:12303-12308 (1997)) Residues a and d of the internal coiled-coil α-helices interact with monomers within that homotrimeric structure. Residues a, d, e, and g of N34 are identified in FIG. 1a and Table 3. In contrast, residues a and d of the external C-terminal helices pack against the e and g residues of the N34 internal trimeric coiled-coil. Id. For residues a and d of the external C-terminal helices see FIG. 1a and Table 4.

TABLE 3
Internal Residues of the N34 Domain

| Residue Number | Amino Acid | Helical Wheel Position |
|---|---|---|
| 38 | I | d |
| 39 | V | e |
| 41 | Q | g |
| 42 | Q | a |
| 45 | L | d |
| 46 | L | e |
| 48 | A | g |
| 49 | I | a |
| 52 | Q | d |
| 53 | Q | e |
| 55 | L | g |
| 56 | L | a |
| 59 | T | d |
| 60 | V | e |
| 62 | G | g |
| 63 | I | a |
| 66 | L | d |
| 67 | Q | e |
| 69 | R | g |

TABLE 4
Internal Residues of the C28 Domain

| Residue Number | Amino Acid | Helical Wheel Position |
|---|---|---|
| 76 | W | a |
| 79 | W | d |
| 83 | I | a |
| 86 | Y | d |
| 90 | I | a |
| 93 | L | d |
| 97 | S | a |
| 100 | Q | d |

The six helix bundle also includes a flexible linker. The length of the linker is not critical for the structure of the molecule, so long as the linker is flexible enough to allow the amino acid chain to loop out and form a hairpin so the external helices can drape over the internal trimeric coiled-coil domain. For example, a six amino acid linker was sufficient to allow interaction between internal and external coiled-coil domains in the model protein, N34-L6-C28. (Tan et al., Proc. Natl. Acad. Sci. U.S.A., 94:12303-12308 (1997)). In the full length gp41 protein the linker is 26 amino acids long. (Louis et al., J. Biol. Chem. 276:29485-29489 (2001)).

The number of helices in a six helix bundle is not critical. For example, GCN4, a yeast protein, forms a trimeric coiled-coil with just three helices and could be used in place of the N34-L6-C28 protein just described. The GCN4 trimeric coiled-coil is described in Weissenhorn et al., Nature 387:426-430 (1997). N35_{CCG}-N13 is stabilized through attachment to a second N-terminal gp41 domain. N34_{CCG} does not include a second attached bundle domain.

2. Relationship Between Conservation of Amino Acid Sequence and Structure of the Six Helix Bundle Domain.

The six helix bundle serves as a scaffold for presentation of the exposed trimeric coiled coil domain. The role of the domain is primarily structural. Thus, for any given six helix polypeptide with the appropriate structure, amino acid sequence can be changed, so long as the structure is maintained. Again, to preserve the structure, only 30% sequence identity between the domain and a divergent amino acid sequence is required. (Chothia and Lesk, EMBO J. 5:823-826, (1986)).

Although the claimed embodiment uses a six helix bundle from HIV gp41 to provide a scaffold, other proteins have similar domains that may be used to present an exposed trimeric coiled-coil domain. Any number of proteins may be used to provide the necessary structure, including but not limited to gp41 from SWV, gp41 from HIV2, yeast GCN4 protein, and GP2 from Ebola virus. (For a review, see Dutch et al., Biosci. Reports 20:597-612 (2000)).

D. Enhancing the Solubility of Exposed Trimeric Coiled-coil Domains.

The claimed embodiment of the present invention is fully soluble only at low pH, e.g. less than pH 4.0. To enhance solubility of the invention at neutral pH, the following strategies can be employed: mutagenesis of charged amino acids on the surface of the protein, addition of polylysine residues to offset the negative charges of the molecule at neutral pH, addition of a lipid binding sequence to facilitate delivery of the molecule in liposomes, and substitution of a six helix bundle domain from a protein other than HIV1 gp41. While N_{CCG}-gp41 is given as an example, similar approaches can be used to increase the solubility of other proteins that contain an exposed trimeric coiled-coil domain from gp41, e.g., N35_{CCG}-N13 and N34CCG.

1. Mutagenesis of Charged Amino Acids on the Surface of the Protein.

N_{CCG}-gp41 is fully soluble and trimeric at low pH (below 3.5), but aggregates above pH 3.5 and precipitates out of solution at neutral pH. Since solubility is affected by pH, and since the only relevant ionizing groups around pH 3-4 are carboxylate groups of glutamate and aspartate residues, it follows that aggregation is the result of electrostatic interactions between negatively charged glutamate or aspartate residues on the surface of N_{CCG}-gp41 with positively charged residues, lysine or arginine. Therefore, by systematically mutating these surface residues it will be possible to engineer a N_{CCG}-gp41 homologue that retains activity but is fully soluble. Surface residues that can be mutated are located both on N35 and C28, so if mutations in C28 alone are sufficient, the sequence of N35 would remain unchanged. Again, surface amino acids are found at helical wheel positions b, c,f, e, and g. C28 and N35 residues that are candidates for mutation are shown in Table 5. Similar mutations can be made to other proteins comprising an exposed trimeric coiled-coil from gp41.

TABLE 5
Charged Surface Residues of the N35 and C28 Domains

| | | | Helical Wheel |
|---|---|---|---|
| Domain | Residue Number | Amino Acid | Position |
| N35 | 12 | R | f |
| N35 | 15 | E | b |
| N35 | 29 | K | b |
| N35 | 34 | R | g |
| C28 | 78 | E | c |
| C28 | 80 | D | e |
| C28 | 81 | R | f |
| C28 | 82 | E | g |
| C28 | 95 | E | f |
| C28 | 96 | E | g |
| C28 | 102 | E | f |
| C28 | 103 | K | g |

2. Addition of Polylysine to the Polypeptide.

The overall charge of the molecule can be changed by adding charged residues to either end of the molecule.

3. Addition of a Lipid Binding Leader Sequence to Facilitate Liposomal Deliver.

Proteins are less likely to aggregate if they are encapsulated in liposomes. Encapsulation into liposomes can be facilitated by addition of a lipid binding leader sequence to either end of the N_{CCG}-gp41 monomers. Lipid binding sequences are known to those of skill in the art.

4. Substitution of a Different Six Helix Bundle Domain.

The six helix bundle domain provides structure but does not serve a functional role in the present invention. Thus, the six helix bundle domain of N_{CCG}-gp41 can be replaced with a six helix bundle domain with different surface residues, which could affect the solubility of the molecule. In addition a three helix bundle domain can be attached to the exposed trimeric coiled-coil as in N35_{CCG}-N13, or no additional bundle domain can used as in N34_{CCG}.

III. Synthesis of Monomeric Subunits

A. Methods to Make Subunits of the Trimeric Protein Complex from DNA Encoding the Protein.

One of skill in the art will recognize that the engineered protein molecules encompassed by the present invention can easily be designed and synthesized using DNA encoding the proteins as the starting material.

1. De Novo Synthesis of DNA Encoding the Monomer.

One of skill in the art will recognize that the monomeric subunits of the trimeric protein complex can be synthesized from DNA molecules that encode the monomeric polypeptide subunits. Depending on the size and desired characteristics of the protein complex, DNA molecules encoding the monomeric subunits can be synthesized de novo. For example, complementary oligonucleotides can be synthesized and annealed to provide a double stranded DNA molecule encoding the monomeric subunits. The single stranded oligos can also be designed to include overhanging cohesive ends that allow the double stranded DNA molecules to be easily ligated. When designing the oligonucleotides, preferred features, such as codon optimization for the production host, and convenient restriction sites, can be engineered into the DNA molecule. In addition, if the subunit contains domains with identical or nearly identical amino acid sequences, different DNA sequences can be used to encode identical protein domains, allowing mutagenesis of each domain separately.

2. Use of Naturally Occurring DNA to Encode Monomers.

Alternatively, DNA sequences may be selected from naturally occurring genes that encode monomeric subunits of a trimeric protein complex. If necessary, the naturally occurring genes can be further modified to suit the needs of the user. One of skill in the art will recognize that PCR and mutagenesis techniques can be used to manipulate a DNA sequence to add convenient restriction sites or to mutagenize a DNA sequence as desired. Detailed descriptions of PCR and mutagenesis techniques can be found, for example at Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al, eds., 1994)). In addition, mutagenesis kits are commercially available.

3. Expression of Cloned Genes Encoding Monomers.

To obtain high level expression of a cloned gene, such as those DNA sequences that encode a monomeric subunit of the N-terminal coiled-coil domain, one typically subclones the DNA sequence into an expression vector that contains a strong promoter to direct transcription, a transcription/translation terminator, and if for a nucleic acid encoding a protein, a ribosome binding site for translational initiation. Suitable bacterial promoters are well known in the art and described, e.g., in Sambrook et al., and Ausubel et al, supra. Bacterial expression systems are available in, e.g., E. coli, Bacillus sp., and Salmonella (Palva et al., Gene 22:229-235 (1983); Mosbach et al., Nature 302:543-545 (1983)). Kits for such expression systems are commercially available. Eukaryotic expression systems for mammalian cells, yeast, and insect cells are well known in the art and are also commercially available.

Selection of the promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the monomeric subunit encoding nucleic acid in host cells. A typical expression cassette thus contains a promoter operably linked to the nucleic acid sequence encoding a monomeric subunit and signals required for efficient polyadenylation of the transcript, ribosome binding sites, and translation termination. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as MBP, GST, and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Expression of proteins from eukaryotic vectors can be also be regulated using inducible promoters. With inducible promoters, expression levels are tied to the concentration of inducing agents, such as tetracycline or ecdysone, by the incorporation of response elements for these agents into the promoter. Generally, high level expression is obtained from inducible promoters only in the presence of the inducing agent; basal expression levels are minimal. Inducible expression vectors are often chosen if expression of the protein of interest is detrimental to eukaryotic cells.

Some expression systems have markers that provide gene amplification such as thymidine kinase and dihydrofolate reductase. Alternatively, high yield expression systems not involving gene amplification are also suitable, such as using a baculovirus vector in insect cells, with a monomeric subunit encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells, if necessary.

Standard transfection methods are used to produce bacterial, mammalian, yeast or insect cell lines that express large quantities of monomeric subunit protein, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well-known procedures for introducing foreign nucleotide sequences into host cells may be used. These include the use of calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the monomeric subunit.

After the expression vector is introduced into the cells, the transfected cells are cultured under conditions favoring expression of the monomeric subunit, which is recovered from the culture using standard techniques identified below.

B. Methods to Chemically Synthesize Monomers.

In addition to the foregoing recombinant techniques, the polypeptides of the invention are optionally synthetically prepared via a wide variety of well-known techniques. Polypeptides of relatively short size are typically synthesized in solution or on a solid support in accordance with conventional techniques (see, e.g., Merrifield, Am. Chem. Soc. 85:2149-2154 (1963)). various automatic synthesizers and sequencers are commercially available and can be used in accordance with known protocols (see, e.g., Stewart & Young, Solid Phase Peptide Synthesis (2nd ed. 1984)). Solid phase synthesis in which the C-terminal amino acid of the sequence is attached to an insoluble support followed by sequential addition of the remaining amino acids in the sequence is the preferred method for the chemical synthesis of the polypeptides of this invention. Techniques for solid phase synthesis are described by Barany & Merrifield, Solid-Phase Peptide Synthesis; pp.3-284 in The Peptides: Analysis, Synthesis, Biology. Vol. 2: Special Methods in Peptide Synthesis, Part A.; Merrifield et al., J. Am. Chem. Soc. 85:2149-2156 (1963); and Stewart et al., Solid Phase Peptide Synthesis (2nd ed. 1984).

C. Purification of Expressed Proteins.

Monomeric subunits of N_{CCG}-gp41 or other proteins comprising N-terminal coiled-coil domains fom gp41 can be purified from any suitable expression system. The monomers may be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al., supra).

1. Purification of Monomers From Recombinant Bacteria

Recombinant monomers can be expressed by transformed bacteria in large amounts, typically after promoter induction; but expression can be constitutive. Promoter induction with IPTG is one example of an inducible promoter system. Bacteria are grown according to standard procedures in the art. Fresh or frozen bacteria cells are used for isolation of protein. Proteins expressed in bacteria may form insoluble aggregates (“inclusion bodies”). Several protocols are suitable for purification of the monomers from inclusion bodies. For example, purification of inclusion bodies typically involves the extraction, separation and/or purification of inclusion bodies by disruption of bacterial cells. The cell suspension can be lysed using 2-3 passages through a French Press; homogenized using a Polytron (Brinkinan Instruments); disrupted enzymatically, e.g., by using lysozyme; or sonicated on ice. Alternate methods of lysing bacteria are apparent to those of skill in the art (see, e.g., Sambrook et al., supra; Ausubel et al., supra).

If necessary, the inclusion bodies are solubilized, and the lysed cell suspension is typically centrifuged to remove unwanted insoluble matter. Proteins that formed the inclusion bodies may be renatured by dilution or dialysis with a compatible buffer. Suitable solvents include, but are not limited to urea (from about 4 M to about 8 M), formamide (at least about 80%, volume/volume basis), and guanidine hydrochloride (from about 4 M to about 8 M). Some solvents which are capable of solubilizing aggregate-forning proteins, for example SDS (sodium dodecyl sulfate), 70% formic acid, are inappropriate for use in this procedure due to the possibility of irreversible denaturation of the proteins, accompanied by a lack of immunogenicity and/or activity.

Although guanidine hydrochloride and similar agents are denaturants, this denaturation is not irreversible and renaturation may occur upon removal (by dialysis, for example) or dilution of the denaturant, allowing re-formation of immunologically and/or biologically active protein. Other suitable buffers are known to those skilled in the art. One of skill in the art will recognize that optimal conditions for renaturation must be chosen for each protein. For example, if a protein is soluble only at low pH, renaturation can be done at low pH. Renaturation conditions can thus be adjusted for proteins with different solubility characteristics. i.e., Proteins that are soluble at neutral pH can be renatured at neutral pH. Monomers are separated from other bacterial proteins by standard separation techniques.

2. Standard Protein Separation Techniques for Purifying Monomers

a. Solubility Fractionation

Often as an initial step, particularly if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant amrnonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

b. Size Differential Filtration

The molecular weight of the monomers can be used to isolate it from proteins of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

c. Column Chromatography

The monomers can also be separated from other proteins on the basis of its size, net surface charge, hydrophobicity, and affinity for ligands. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

IV. Assays for Function of an Exposed Trimeric Coiled-coil Domain From gp41

Properly folded exposed trimeric coiled-coil domains from gp41, such as N_{CCG}-gp41, inhibit membrane fusion mediated through the HIV gp41 protein. Assays of membrane fusion can thus be used to determine the activity of the trimeric polypeptide complex. The fusion event can take place between cells engineered to express portions of the fusion machinery, or between cells infected with the HIV virus and uninfected cells, or between live HIV virus and cells, either in vitro or in vivo. The term cell fusion is used interchangeably with the term membrane fusion.

One of skill in the art will recognize that the function of an exposed trimeric coiled-coil domain from gp4lcan be verified using the assays described below. Verification of the function of N_{CCG}-gp41 is useful when the amino acid sequence of N_{CCG}-gp41 has been altered from that of the prototype or where different combination of proteins domains have been assembled.

A. Assays of Inhibition of HIV-induced Membrane Fusion.

1. Transcriptional Activation of a Reporter Gene.

Transcriptional activation can be used to assay cellular fusion between cells engineered to express HIV fusion machinery. Briefly, target cells are transduced with a vector to express a CD4 co-receptor and a reporter gene under regulable control. For example, the E. coli LacZ gene can be expressed on a plasmid such that transcription can occur only in the presence of the T7 RNA polymerase. Effector cells are then transduced to express the HIV Env protein and the bacteriophage T7 RNA polymerase. Target and effector cells are mixed in the presence of soluble CD4 protein. Expression of the reporter gene, LacZ, will occur only if the two cell types fuse, permitting the bacteriophage T7 RNA polymerase to activate expression of LacZ. Activity of the LacZ gene product can then be used to quantify cellular fusion.

To assay the function of an exposed trimeric coiled-coil domain from gp41, the cells are incubated in the presence of the exposed trimeric coiled-coil after mixing with soluble CD4 protein. A range of protein concentrations can be tested and appropriate control reactions should be included.

One of skill in the art will recognize that a variety of regulable expression systems can be used in the assay, as well as a variety of reporter genes. In addition, because the assay provides the HIV fusion machinery, the particular cell type used in the assay is not critical so long as the cells are able to be transduced by some method (E.g., Transient or stable transfection, infection by virus and other techniques can be used to introduce foreign DNA into the cells.)

A variation ofthis technique can be used to produce HIV virus engineered to express a reporter gene. Cell fusion mediated by the engineered virus can then be assayed by detection of the reporter gene. (See, for example Chen, et al. J. Virol. 68:654-660 (1994); Chan etal., Proc. Natl. Acad. Sci. USA, 95:15613 (1998)).

2. Syncytia Formation.

A cell fusion assay can be utilized to test the peptides' ability to inhibit viral-induced syncytia formation in vitro. (Chan et al., Proc. Natl.. Acad. Sci. USA, 95:15613 (1998)). Uninfected cells are incubated in the presence of cells chronically infected with a HIV and a polypeptide to be assayed. For each polypeptide, a range of concentrations can be tested. Appropriate controls can also be included in the experiment. Standard cell culture conditions are used and are well known to those of ordinary skill in the art. After incubation for an appropriate period (24 hours at 37° C., for example) the culture is examined microscopically for the presence of multinucleated giant cells, indicative of cell fusion and syncytia formation. Well-known stains, such as crystal violet stain, may be used to facilitate syncytial visualization. Taking HIV as an example, such an assay would comprise CD4+ cells (such as Molt or CEM cells, for example) cultured in the presence of chronically HIV-infected cells and a polypeptide to be assayed.

Syncytia formation can also be tested using live HIV virus as a starting material. HIV virus is used to infect appropriate cell lines (usually CD4+) and active HIV will cause syncytia formation in those cell lines. (See for example, Shibata et al., J. Virol. 69:4453-4462 (1995)). Polypeptides of interest can be tested for inhibition of syncytial formation after infection by live virus as described above.

3. Reverse Transcriptase Activity.

The ability of the trimeric protein complex to inhibit HIV infection can also be assayed using the retroviral enzyme reverse transcriptase. The assay can be done in vitro or in vivo.

For the in vitro assay, an appropriate concentration (i.e., TCID.sub.50) of virus is incubated with CD4⁺ cells in the presence of the trimeric complex to be tested. A range of complex concentrations can be used and appropriate controls can be included. After incubation for an appropriate period (e.g., 7 days) of culturing, a cell-free supematant is prepared, using standard procedures, and tested for the present of reverse transcriptase (RT) activity as a measure of successful infection. The RT activity may be tested using standard techniques such as those described by, for example, Goff et al. (Goff, S. et al., J. Virol. 38:239-248 (1981)) and/or Willey et al. (Willey, R. et al., J. Virol. 62:139-147 (1988)). In vivo assays may also be utilized to test, for example, the antiviral activity or vaccine activity of the peptides of the invention. To test for anti-HIV activity, for example, the in vivo model described in Barnett et al. (Barnett, S. W. et al., Science 266:642-646 (1994)) may be used.

V. Biophysical Characteristics of a Trimeric Protein Complex

The present invention includes amino acid sequences that are predicted to form particular structures, to adopt particular conformations, and to form stable multisubunit complexes. One of skill in the art will recognize that formation of the predicted structures and complexes can be verified using standard techniques.

A. Verification of Protein Structure.

One of skill in the art will recognize that there are many methods to determine the three dimensional structure of a protein. The techniques include, but are not limited to circular dichroism, NMR, X-ray crystallography, and computer modeling.

1. Circular Dichroism

Circular dichroism (CD) is a standard spectroscopic technique which can be reliably used to evaluate the % helical content of a protein (Andrade et al., Protein Eng. 6:383-390 (1993)).

2. NMR

Nuclear magnetic resonance (NMR) is a technique that can be used to determine the three-dimensional structures of proteins in solution. The main source of structural information comprises short interproton distances derived from nuclear Overhauser enhancement measurements, but can be supplemented by torsion angle restraints derived from three-bond coupling constants and chemical shift data, and by orientational restraints derived from dipolar couplings. (Clore & Gronenborn, Trends in Biotech. 16:22-34 (1998)).

3. X-ray Crystallography

X-ray crystallography is a technique used to determine the three-dimensional structures of molecules in the crystal state. Fourier transformation of a diffraction pattern yields an electron density map that can be interpreted in terms of an atomic model. (Drenth, Principles of Protein X-ray crystallography, Springer-Verlag, New York (1994)).

4. Computer Modeling

Computer modeling is a technique that can be used to model related structures based on known three-dimensional structures of homologous molecules. Standard software is commercially available. (See www.accelrys.com for the multitude of software available to do computer modeling.)

B. Formation of Trimeric Complex and Detection of Disulfide Bonds.

The present invention is predicted to form stable multimeric complexes through covalent bonding at engineered disulfide bridges.

1. Measurement of Mass.

The present invention provides polypeptide monomers that form functional trimeric complexes. Measurement of molecular mass can be used to verify formation of a trimeric polypeptide complex. For example, a homotrimeric complex should have a molecular mass three times that predicted for a monomeric subunit.

a. SDS-PAGE

SDS polyacrylamide gel electrophoresis (SDS-PAGE) is used to measure molecular mass. Proteins are denatured by boiling in the presence of the detergent sodium dodecyl sulfate (SDS) and, if desired, the reducing agent mercaptoethanol. The mercaptoethanol oxidizes any disulfide bonds, thereby separating polypeptides that were covalently linked through a disulfide bond and not through a peptide bond. SDS denatures proteins by binding to hydrophobic regions. The total number of SDS molecules bound to the protein is proportional to the polypeptide length of the molecule, or its molecular mass.

Proteins are separated on a polyacrylamide gel on the basis of charge. Each bound SDS molecule provides two negative charges masking the protein's native charge. Because the total number of SDS molecules bound to the protein is proportional to the protein's weight, the protein's charge is also proportional to its molecular weight which can thus be determined by its migration behavior on the gel. (Zubay, Biochemistry, 1986.)

b. Size Exclusion Chromatography

Size exclusion chromatography, also known as gel filtration chromatography, separates proteins on the basis of size as they pass through a porous solid matrix. The matrix is made up of beads with very small passages through them. Large proteins are excluded from the beads and exit the column first. Smaller proteins are retained by the porous beads and elute last. The technique can be used to quantitate an unknown molecular weight of a protein by including proteins of known molecular weight in the sample and comparing their elution profile to that of the unknown protein. (Zubay, Biochemistry, 1986.)

c. Sedimentation Equilibrium

Sedimentation equilibrium: Sedimentation equilibrium by analytical ultracentrifugation is a standard biophysical technique used to determine the molecular weight of biomolecules. (See e. g., Cantor & Schimmel, Biophysical Chemistry, W.H. Freeman & Co, (1980); Zubay, Biochemistry, (1986.)). Protein molecules are denser than water, and can be made to sediment out of water at very high centrifugal force fields. At a certain point the protein molecules will stop migrating toward the bottom of the centrifuge tube as diffusion forces begin to counterbalance the downward sedimentation forces. The molecular weight of a protein can be determined by comparing its sedimentation coefficient to that of a protein of known molecular weight. Alternatively, commercially available software can be used to analyze the data (Optima XL-A data analysis software, Beckman).

d. Mass Spec

Mass spectrometry is a standard technology used to determine the molecular mass of any molecules. Current standard equipment can readily determine masses to within less than 1 atomic mass units.

2. Stabilization of the Trimeric Complex Through Disulfide Bonds

Disulfide bonds are formed by oxidation of cysteine residues and are disrupted by reducing agents, such as 2-mercaptoethanol or dithiothreitol. Assuming appropriate renaturation conditions have been used during purification of the trimeric polypeptide complex, the complex can be stabilized by the formation of disulfide bonds between monomeric subunits. Disulfide bonds can be detected by determining the molecular mass of the protein of interest in the presence or absence of reducing agents. In the absence of reducing agents the trimeric complex will have a molecular weight expected for the trimeric complex on SDS-PAGE. In the presence of a reducing agent disulfide bonds will be disrupted and the protein will be primarily in the monomeric form. Thus, in the presence of a reducing agent the protein will have the weight of the monomeric form when analyzed using SDS-PAGE.

VI. Use of an Exposed Trimeric Coiled-coil Domain From gp41 as an HIV Vaccine

The present invention encompasses trimeric polypeptide complexes engineered to present a stable and exposed trimeric coiled-coil domains from gp41. The invention presents conformational epitopes suitable for use as a vaccine to prevent infection by the HIV virus. The preparation of vaccines which contain an immunogenic polypeptide(s) as an active ingredient(s) is known to one skilled in the art. Typically, such vaccines are prepared as injectables, either as liquid solutions or suspensions; solid forms suitable for solution in, or suspension in, liquid prior to injection may also be prepared. the preparation may also be emulsified, or the polypeptide(s) encapsulated in liposomes. The active immunogenic ingredients are often mixed with excipients which are pharmaceutically acceptable and compatible with the active ingredient. Suitable excipients are, for example, water, saline, dextrose, glycerol, ethanol, or the like and combinations thereof. In addition, if desired, the vaccine may contain minor amounts of auxiliary substances such as wetting or emulsifying agents, pH buffering agents, and/or adjuvants which enhance the effectiveness of the vaccine. Examples of adjuvants which may be effective include, but are not limited to: aluminum hydroxide, N-acetyl-muramyl-L-threonyl-D-isoglutamine (thr-MDP), N-acetyl-nor-muramyl-L-alanyl-D-isoglutamine (CGP 11637), referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE, and RIBI, which contains three components extracted from bacteria, monophosphoryl lipid A, trehalose dimycolate and cell wall skeleton (MPL+TDM+CWS) in a 2% squalene/Tween 80 emulsion. The effectiveness of an adjuvant may be determined by measuring the amount of antibodies directed against a protein of interest, the antibodies resulting from administration of this polypeptide in vaccines which are also comprised of the various adjuvants.

The immunogenic proteins can be formulated into the vaccine as neutral or salt forms. Pharmaceutically acceptable salts include the acid addition salts (formed with free amino groups of the peptide) and which are formed with inorganic acids such as, for example, hydrochloric or phosphoric acids, or organic acids such as acetic, oxalic, tartaric, rmaleic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The vaccines are conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkylene glycols or triglycerides; such suppositories may be formed from mixtures containing the active ingredient in the range of 0.5% to 10%, preferably 1%-2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10%-95% of active ingredient, preferably 25%-70%.

In addition to the above, it is also possible to prepare live vaccines of attenuated microorganisms which express recombinant polypeptides of interest. Suitable attenuated microorganisms are known in the art and include, for example, viruses (e.g., vaccinia virus) as well as bacteria.

The vaccines are administered in a manner compatible with the dosage formulation, and in such amount as will be prophylactically and/or therapeutically effective. The quantity to be administered, which is generally in the range of 5 μg to 250 μg of antigen per dose, depends on the subject to be treated, capacity of the subject's immune system to synthesize antibodies, and the degree of protection desired. Precise amounts of active ingredient required to be administered may depend on the judgment of the practitioner and may be peculiar to each individual.

The vaccine can be given in a single dose schedule, or preferably in a multiple dose schedule. A multiple dose schedule is one in which a primary course of vaccination may be with 1-10 separate doses, followed by other doses given at subsequent time intervals required to maintain and/or reinforce the immune response, for example, at 1-4 months for a second dose, and if needed, a subsequent dose(s) after several months. The dosage regimen will also, at least in part, be determined by the need of the individual and be dependent upon the judgment of the practitioner.

In addition, the vaccine containing the antigen sets comprised of an exposed trimeric coiled-coil domain from gp41 described above, can be administered in conjunction with other immunoregulatory agents, for example, immune globulins.

The compositions of the present invention can be administered to individuals to generate polyclonal antibodies (purified or isolated from serum using conventional techniques) which can then be used in a number of applications. For example, the polyclonal antibodies can be used to passively immunize an individual, or as immunochemical reagents.

VII. Pharmaceutical Compositions Including Exposed Trimeric Coiled-coil Domains From gp41

Infection by and dissemination of the HIV virus proceeds through virus-cell or cell-cell fusion mediated through the HIV gp41 protein. The present invention, encompases trimeric polypeptide complexes engineered to present a stable and exposed trimeric coiled-coil doamins from gp41. For example, N_{CCG}-gp41, N35_{CCG}-N13, and N34_{CCG} inhibit membrane fusion at nanomolar concentrations, presumably by binding to the C-terminal helices of gp41 in the pre-hairpin intermediate. A pharmaceutical composition including any one of those proteins or a related protein could be used to block fusion mediated by gp41 in subjects infected with HIV.

Pharmaceutically acceptable carriers are determined in part by the particular composition being administered (e.g., nucleic acid, protein, modulatory compounds or transduced cell), as well as by the particular method used to administer the composition. Accordingly, there are a wide variety of suitable formulations of pharmaceutical compositions of the present invention (see, e.g., Remington 's Pharmaceutical Sciences, 17^{th} ed., 1989). Administration can be in any convenient manner, e.g., by injection, oral administration, inhalation, transdermal application, or rectal administration.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The compound of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration and intravenous administration are the preferred methods of administration. The formulations of commends can be presented in unit-dose or multi-dose sealed containers, such as ampules and vials.

Injection solutions and suspensions can be prepared from sterile powders, granules, and tablets of the kind previously described.

In therapeutic applications, the exposed trimeric coiled-coil domains of the invention are administered to a patient in an amount sufficient to prevent HIV mediated membrane fusion, and spread of the virus. An amount adequate to accomplish this is defined as a “therapeutically effective dose.” Amounts effective for this use will depend on, for example, the manner of administration, the weight and general state of health of the patient, and the judgment of the prescribing physician. For example, for the prevention of HIV mediated membrane fusion and spread of the virus an amount of exposed trimeric coiled-coil domain from gp41 falling within the range of 10 mg to 200 mg given once a day would be a therapeutically effective amount.

VIII. Therapeutic Use of Antibodies Directed Against the Exposed Trimeric Coiled-coil Domain From gp41

The present invention includes an exposed trimeric coiled-coil domain from gp41 that presents conformational epitopes suitable to generate antibodies directed against the pre-hairpin fusion intermediate. Antibody binding to the exposed N-terminal coiled-coil domain will block membrane fusion and limit the dissemination of the HIV virus within a subject infected with the virus.

A. Antibodies to the Exposed Trimeric Coiled-coil Domain From gp41

Methods of producing polyclonal and monoclonal antibodies that react specifically with an exposed trimeric coiled-coil domain from gp41 are known to those of skill in the art (see, e.g., Coligan, Current Protocols in Immunology (1991); Harlow & Lane, supra; Goding, Monoclonal Antibodies: Principles and Practice (2d ed. 1986); and Kohler & Milstein, Nature 256:495-497 (1975). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)). Methods of production of polyclonal antibodies are known to those of skill in the art. An inbred strain of mice (e.g., BALB/C mice) or rabbits is immunized with the protein using a standard adjuvant, such as Freund's adjuvant, and a standard immunization protocol. The animal's immune response to the immunogen preparation is monitored by taking test bleeds and determining the titer of reactivity to the beta subunits. When appropriately high titers of antibody to the immunogen are obtained, blood is collected from the animal and antisera are prepared. Further fractionation of the antisera to enrich for antibodies reactive to the protein can be done if desired (see, Harlow & Lane, supra).

Monoclonal antibodies maybe obtained by various techniques familiar to those skilled in the art. Briefly, spleen cells from an animal immunized with a desired antigen are immortalized, commonly by fision with a myeloma cell (see, Kohler & Milstein, Eur. J. Immunol. 6:511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse, et al., Science 246:1275-1281 (1989).

Monoclonal antibodies and polyclonal sera are collected and titered against the immunogen protein in an immunoassay, for example, a solid phase immunoassay with the immunogen immobilized on a solid support. Typically, polyclonal antisera with a titer of 10⁴ or greater are selected and tested for their cross reactivity against non-N35 proteins, using a competitive binding immunoassay. Specific polyclonal antisera and monoclonal antibodies will usually bind with a K_{d} of at least about 0.1 mM, more usually at least about 1 μM, preferably at least about 0.1 μM or better, and most preferably, 0.01 μM or better. Antibodies specific only for an N35 domain, can also be made, by subtracting out antibodies directed against the six-helix bundle, for example.

Once the specific antibodies against the exposed trimeric coiled-coil domain from gp41 are available, the antibodies can be used for passive immunization. In addition, the antibodies can be used to detect trimeric coiled-coil containing proteins, including including the engineered proteins of the invention, by a variety of immunoassay methods. For a review of immunological and immunoassay procedures, see Basic and Clinical Immunology (Stites & Terr eds., 7^{th} ed. 1991). Moreover, the immunoassays of the present invention can be performed in any of several configurations, which are reviewed extensively in Enzyme Immunoassay (Maggio, ed., 1980); and Harlow & Lane, supra.

B. Passive Immunization.

HIV has been disclosed as treatable using passive immunization. See for example Jackson et al., Lancet, 17:647-652, (1988); Karpas et al., Proc. Natl. Acad. Sci., USA, 87:7613-7616 (1990), Eichberg, J. W., K. K. Murthy, R. H. Ward, and A. M. Prince. 1992. Prevention of HIV infection by passive immunization with HIVIG or CD4-IgG. AIDS Res. Hum. Retroviruses 8:1515 and U.S. Pat. No. 5,830,476 entitled “Active induction or passive inmuunization of anti-Gp48 antibodies and isolated gp48 protein”.

Passive immunization can be accomplished with polyclonal antibodies, monoclonal antibodies, or antibody fragments.

In one embodiment, the passive immunization method comprises administering a composition comprising more than one species of human monoclonal antibody of this invention, preferably directed to non-competing epitopes or directed to distinct serotypes or strains of HIV, as to afford increased effectiveness of the passive immunotherapy.

A therapeutically (immunotherapeutically) effective amount of a humanized or human antibody is a predetermined amount calculated to achieve the desired effect, i.e., to neutralize the HIV present in the sample or in the patient, and thereby decrease the amount of detectable HIV in the sample or patient. In the case of in vivo therapies of persons already infected, an effective amount can be measured by improvements in one or more symptoms associated with HIV-induced disease occurring in the patient, or by serological decreases in HIV antigens.

Thus, the relevant dosage ranges for the administration of the monoclonal or other antibodies of the invention are those large enough to produce the desired effect in which the symptoms of the HIV disease are ameliorated or the likelihood of infection decreased. The dosage should not be so large as to cause adverse side effects, such as hyperviscosity syndromes, pulmonary edema, congestive heart failure, and the like. Generally, the dosage will vary with the age, condition, sex and extent of the disease in the patient and can be determined by one of skill in the art. The dosage can be adjusted by the individual physician in the event of any complication.

A therapeutically effective amount of an antibody of this invention is typically an amount of antibody such that when administered in a physiologically tolerable composition is sufficient to achieve a plasma concentration of from about 0.1 microgram (μg) per milliliter (ml) to about 100 μg/ml, preferably from about 1 μg/ml to about 5 μg/ml, and usually about 5 μg/ml. Stated differently, the dosage can vary from about 0.1 mg/kg to about 300 mg/kg, preferably from about 0.2 mg/kg to about 200 mg/kg, most preferably from-about 0.5 mg/kg to about 20 mg/kg, in one or more dose administrations daily, for one or several days.

The antibodies of the invention can be administered parenterally by injection or by gradual infusion over time. Although the HIV infection is typically systemic and therefore most often treated by intravenous administration of therapeutic compositions, other tissues and delivery means are contemplated where there is a likelihood that the tissue targeted contains infectious HIV. Thus, antibodies of the invention can be administered intravenously, intraperitoneally, intramuscularly, subcutaneously, intracavity, transdermally, and can be delivered by peristaltic means.

The therapeutic compositions containing antibodies of this invention are conventionally administered intravenously, as by injection of a unit dose, for example. The term “unit dose” when used in reference to a therapeutic composition of the present invention refers to physically discrete units suitable as unitary dosage for the subject, each unit containing a predetermined quantity of active material calculated to produce the desired therapeutic effect in association with the required diluent; i.e., carrier, or vehicle.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

### EXAMPLES

The following examples are provided by way of illustration only and not by way of limitation. Those of skill in the art will readily recognize a variety of noncritical parameters that could be changed or modified to yield essentially similar results.

### Example 1

### Chemical Synthesis and Cloning of Nccg-gp41

Design of N-gp41

The foundation of the present invention is the minimal trimeric core of the ectodomain of the gp41 protein. The subunits of the minimal trimeric ectoderm core of gp41 have the following domain structure: An N-terminal coiled-coil domain (N34, SEQ ID NO: 2) linked to the C-terminal alpha helical domain (C28, SEQ ID NO:3) through a six amino acid linker (L6, SEQ ID NO: 19). To construct the claimed species, a second N-terminal coiled-coil domain (N35, SEQ ID NO: 1) was grafted onto the N-terminus of the N34-(L6)-C28 core (SEQ ID NO:16), generating a 69 residue continuous α-helix. Care was taken to continuously maintain the helical register from N35 through N34. This umnutated protein is referred to as N-gp41.

Chemical Synthesis and Cloning of N-gp41

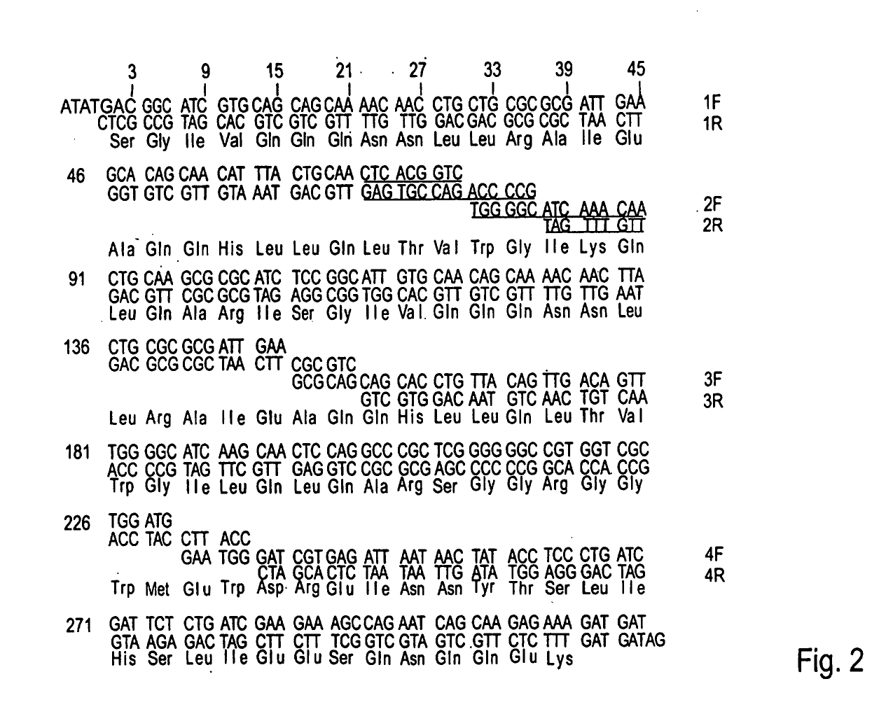

The strategy employed for synthesizing the DNA encoding the N-gp41 protein is shown in FIG. 2. The coding sequence of N-gp41 was synthesized as four single-strand oligonucleotide fragments: 1F(SEQ ID NO:7), 2F(SEQ ID NO:9), 3F(SEQ ID NO: 11) and 4F(SEQ ID NO:13). The complementary sequence of N-gp41 was also synthesized as four single-strand oligonucleotide fragments: 1R(SEQ ID NO:8), 2R(SEQ ID NO:10), 3R(SEQ ID NO: 12) and 4R(SEQ ID NO: 14). These fragments were assembled in a manner similar to that described previously (Louis et al., Biochem. Biophys. Res. Comm., 159:87-94 (1989)). Complementary pairs of oligonucleotides were designed to have 3′ overhangs after annealing to facilitate ligation to an adjoining double strand oligonucleotide. Oligonucleotide 1F included the translational initiation codon, ATG. Oligonucleotide 4F included the stop codon, TAG. The single-strand oligonucleotides were purified by polyacrylamide gel electrophoresis. To generate the full-length coding sequence of N-gp41, each of the oligonucleotides was phosphorylated except for 1F and 4R. Complementary pairs of single-strand oligonucleotides 1F and 1R, 2F and 2R, 3F and 3R, 4F and 4R were annealed and then ligated using the 3′ overhangs. The double-stranded, fuill-length N-gp41 DNA (SEQ ID NO: 15) was isolated by gel electrophoresis and subsequently cloned into the Ndel and BamH1 sites of the pET11a vector (Novagen, Madison, Wis.). The DNA sequence of two individual clones was confirmed by sequencing.

Two features of the nucleotide sequence are noteworthy. First, codon usage was optimized for E. coli. Second, within these confines, non-identical codon usage was employed for the N35 and N34 portions of the gene. Hence, while the amino acid sequences of residues N34 and N35 of N-gp41 are identical (FIG. 1a), the corresponding nucleotide sequences are different (FIG. 2). This allows one to mutagenize residues within N35 without affecting N34.

Structure and Stabilization of N-gp41

The structure of the N34-(L6)-C28 core (SEQ ID NO:16) in its trimeric form has been solved crystallographically (Tan et al., Proc. Natl. Acad. Sci. U.S.A., 94:12303-12308 (1997)). The protein complex forms a thermostable trimer of hairpins in which N34 and C28 are entirely helical and arranged in a six-helix bundle. Because an N35 domain was added to the N-terminus of N34-(L6)-C28, the trimeric structure of N-gp41 will include an exposed trimeric coiled-coil of N35 helices. The trimeric N35 helices are fully accessible after formation and stabilized by covalent linkage to the trimeric N34-(L6)-C28 core.

To further stabilize the trimeric coiled-coil of N35 helices, and to ensure that the entire molecule remains trimeric at very low concentrations, two cysteine residues were introduced into the N35 monomer to facilitate the formation of disulfide linkages between the monomers after formation of the trimeric complex. Leu-Gln-Ala located at the C-terminal end of N35 (residues 31-33 of the construct) to Cys-Cys-Gly (FIGS. 1, a and b). The L31C, Q32C and A33G mutations were introduced into the N-gp41 construct using the Quick-Change mutagenesis protocol (Stratagene, CA). The forward primer was SEQ ID NO: 17; the reverse primer was SEQ ID NO: 18. This methodology was previously used in an attempt to stabilize the gp160 envelope protein (Farzan et al., J. Virol., 72:7620-7625 (1998)). The location of the two cysteine residues on the helix face (at positions d and e, respectively) was chosen so that three intermolecular disulfide bonds can readily be formed between the three subunits. If the three subunits are arbitrarily given the designations A, B, and C, the disulfide bridges would form as follows: Cys31(A)-Cys32(B), Cys31(B)-Cys32(C) and Cys31(C)-Cys32(A) (FIGS. 1c,d). The A33G mutation was employed to ensure that minor adjustments of the polypeptide backbone can readily occur to ensure disulfide bond formation. This cysteine containing monomer is known as N_{CCG}-gp41.

Structural Model for N_{CCG}-gp41

The proposed structure for the chimeric protein, which we term N_{CCG}-gp41, is shown in FIG. 1b. The model was constructed from the X-ray structure of N34-(L6)-C28 (Tan et al., Proc. Natl. Acad. Sci. U.S.A., 94:12303-12308 (1997))and the internal trimeric coiled-coil of N-helices from the X-ray structure of a longer ectodomain fragment of gp41 (comprising residues 541-588 and 628-665 of HIV-1 Env) (Weissenhom et al., Nature, 387:426-430 (1997)). N35 was grafted onto N34 by best-fitting the backbone of residues 581-586 (i.e. 5 residues C-terminal of N35) of the longer HIV gp41 ectodomain onto the backbone of residues 546-551 (i.e. the first 5 residues) of the N34-(L6)-C28 construct, followed by deletion of residues 581-586; substitution of LeuS76, Leu577 and Ala578 in N35 by Cys, Cys and Gly, respectively; and regularization to covalently link the N35 and N34 helices of each subunit and form intermolecular disulfide bonds with good stereochemistry.

According to the model, N35 (residues 1-35 of N_{CCG}-gp4l) and N34 (residues 36-69 of N_{CCG}-gp41) form a continuous 69 residue alpha-helix, approximately 100 Å long. The internal trimeric coiled-coil of N34 helices is surrounded by three C-terminal helices (C28 corresponding to residues 76-103 of N_{CCG}-gp41), while the trimeric coiled-coil of N35 helices, ˜50 Å in length, is fully exposed to solvent.

### Example 2

### Expression, Purification, and Folding of N_{CCG}-g41

N_{CCG}-gp41 was expressed in bacteria, purified as a denatured protein and refolded and allowed to assemble into a trimeric complex. E. coli cells were transformed with the pET11 plasmid that encoded N_{CCG}-gp41 protein. Cells were grown at 37° C. either in Luria-Bertani medium or in a modified minimal medium for uniform (>99%) ¹⁵N labeling with ¹⁵NH₄Cl as the sole nitrogen source. Expression of N_{CCG}-gp41 protein was induced by growth of bacteria in the presence of 2 mM isopropyl β-D-thiogalactoside for four hours.

After harvesting, cells from one liter of bacterial culture were suspended in 20 volumes of buffer A (50 mM Tris-HCl pH 8.2, 10 mM EDTA, and 10 mM DTT). Cells were lysed by sonication at 4° C. in the presence of 100 μg/ml lysozyme. The insoluble fraction, containing the N_{CCG}-gp41 protein, was first resuspended in buffer A containing 1 M urea and 0.5% Triton X-100. Insoluble material was pelleted by centrifugation at 20,000×g for thirty minutes at 4° C. and then resuspended in buffer A alone. Again, the insoluble fraction was pelleted by centrifugation at 4° C. at 20,000×g for thirty minutes. The final pellet was solubilized in 50 mM Tris-HCl, pH 8.0, 7.5 M guanidine-HCl, 5 mM ethylenediaminetetraacetic acid (EDTA), and 20 mnM dithiothreitol (DTT) to yield a protein concentration not exceeding 20 mg/ml.

Denatured N_{CCG}-gp41 protein was purified from the solubilized bacterial pellet by gel filtration chromatography. Thirty milligrams of protein was applied to a Superdex-75 column (HiLoad 2.6 cm×60 cm, Amersham Pharmacia Biotech, Piscataway, N.J.) equilibrated in 50 mM Tris-HCl, pH 8,4 M guanidine-HCl, 5 mM EDTA, and 5 mM DTT. The column was run at ambient temperature with a flow-rate of 3 ml/minute. The protein was further purified using reverse-phase high performance liquid chromotography (HPLC). Peak fractions from the gel filtration column were subjected to reverse-phase HPLC on POROS RII resin (Perceptive Biosystems, MA) using a linear gradient of 0 to 60% acetonitrile/0.05% trifluoroacetic acid (TFA). Peak fractions from HPLC were combined.

The purified N_{CCG}-gp41 protein was refolded by dialysis. Seven milligrams of protein was diluted to a concentration of 0.2 mg/ml in 35% acetonitrile/water/0.05% TFA. The diluted protein was dialyzed against two liters of 50 mM sodium formate buffer, pH 3.0 for three hours. After a buffer change, the sample was dialyzed overnight at 4° C. After dialysis, the protein was concentrated to approximately 3 mg/ml and stored at 4° C.

### Example 3

### Biophysical Properties of N_{CCG}-gp41

According to the model for N_{CCG}-gp41 structure, the polypeptide will form a trimeric complex, disulfide bonds between the monomers will stabilize the trimeric complex, and the trimeric complex will have substantial α-helical content. All three predictions of the model were tested and shown to be correct.

N_{CCG}-gp41 Forms a Trimeric Complex.

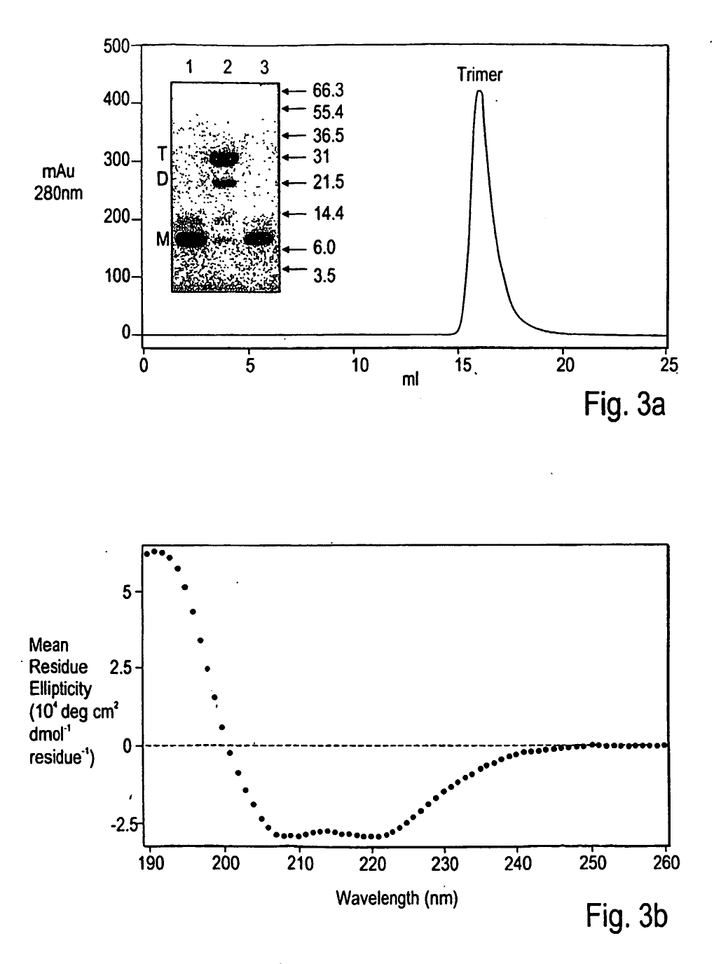

After dialysis the folded N_{CCG}-gp41 is fully soluble at low pH, (less than pH 4) and forms a trimeric complex. Formation of the trimeric complex was shown in a number of ways. When the folded N_{CCG}-gp41 protein was analyzed by SDS-polyacrylamide gel electrophoresis (SDS-PAGE)under nonreducing conditions, about ninety percent of the protein had a molecular weight consistent with that of a trimeric form of the protein (FIG. 3a).

Purified native Ncco-gp41 was also analyzed by size-exclusion column chromatography. N_{CCG}-gp41 was fractionated on a Superdex-200 column in 50 mM sodium formate buffer, pH 3.0 and 0.2 M guanidine-HCl at room temperature. The protein eluted as a single trimeric peak on size-exclusion chromatography (FIG. 3a).

Quantitative analysis of elution profiles from a Superdex-75 column as described (Yang et al., J. Mol. Biol., 288:403-412 (1999)), indicated that N_{CCG}-gp41 elutes with an apparent molecular mass of 30,000 Da. This is similar to the predicted molecular weight of the trimeric form: 35,442 Da. No evidence of dimer or monomer forms was apparent at the lowest concentration of about 140 nM tested on the Superdex-75 column.

The trimeric nature of N_{CCG}-gp41 was confirmed by sedimentation equilibrium studies. Sedimentation equilibrium experiments were conducted at 20° C. and at three different rotor speeds (10,000, 12,000 and 14,000) on a Beckman Optima XL-A analytical ultracentrifuge. Protein samples were prepared in 50 mM sodium formate buffer, pH 3.0, and loaded into the ultracentrifuge cells at nominal loading concentrations of 0.80 A₂₈₀. Data were analyzed in terms of a single ideal solute to obtain the buoyant molecular mass, M(1−νρ), using the Optima XL-A data analysis software (Beckman). The value for the experimental molecular mass M was determined using calculated values for the density ρ (determined at 20° C. using standard tables) and partial specific volume ν (calculated on the basis of amino acid composition (Perkins, Eur. J. Biochem., 157:169-180 (1986)). Results of the sedimentation equilibrium experiments indicated that N_{CCG}-gp41 behaves as a single monodisperse species with a molecular mass of 35,600±150 Da. Again, the value is close to the molecular mass predicted for the trimeric form of the protein: 35,442 Da.

Finally, mass spectroscopic analysis was also used to demonstrate the trimeric nature of N_{CCG}-gp41. Mass spectroscopic analyses of non-reduced N_{CCG}-gp41 showed the presence of trimer and minor dimer forms with experimental masses of m/z 35,442 and 23,629, respectively. These values are essentially identical to the expected values of 35441.7 and 23627.8 for the trimer and dimer, respectively.

The Trimeric Complex of N_{CCG}-gp41 is Stabilized by Disulfide Bonds

N_{CCG}-gp41 was specifically designed with the aim of generating a protein in which the subunits of the trimer are covalently linked by intermolecular disulfide bonds. This is indeed found to be the case experimentally. SDS-PAGE of refolded N_{CCG}-gp41 demonstrates that under non-reducing conditions the majority (˜90%) of the protein migrates as a trimer with about 10% as a dimer (see FIG. 3a, lane 2). After addition of 2-mercaptoethanol all the N_{CCG}-gp41 migrates as a monomer, as predicted by the model (FIG. 3a, lane 2).

The N_{CCG}-gp41 Trimer has Substantial α-helical Content

A circular dichroism (CD) spectrum of N_{CCG}-gp41 was recorded at 25° C. on a JASCO J-720 spectropolarimeter using a 0.05 cm path length cell. The assay was carried out at ambient temperature using 9.94 μM N_{CCG}-gp41 trimer in 10 mM sodium formate buffer, pH 3. Quantitative evaluation of secondary structure from the CD spectrum was done using the program k2d (available at http://bioinformatik.biochemtech.uni-halle.de/) which employs a neural network approach for CD spectra deconvolution (Bohm et al., Protein Eng. 5, 191-195 (1992)).

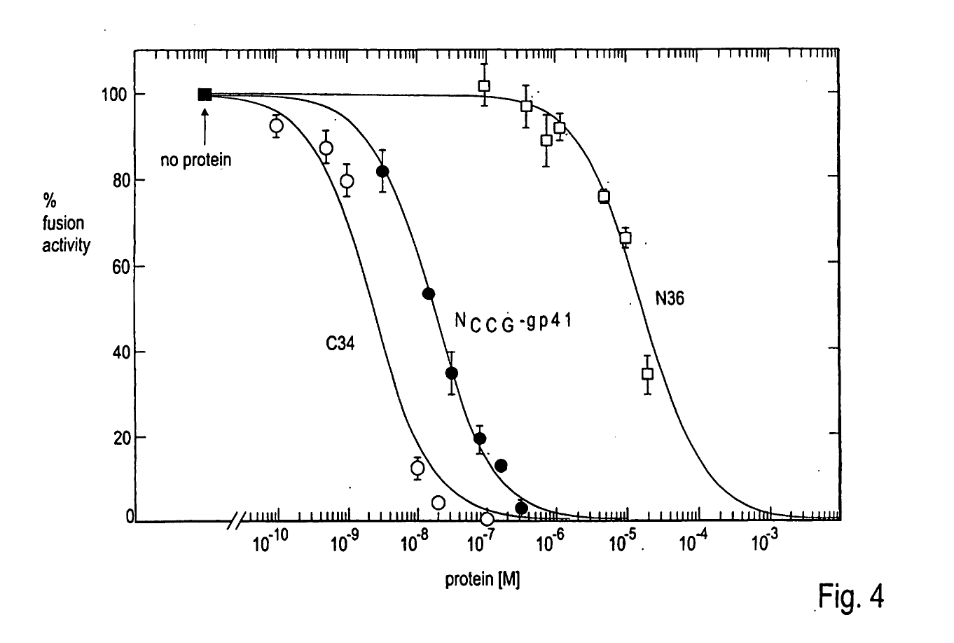

The CD spectrum of N_{CCG}-gp41 (FIG. 4) displayed the characteristic signature of an α-helical protein with double minimae at 208 and 222 nm. Deconvolution of the CD spectrum with the neural network program k2d (Id.) yields an α-helical content of 96%. The only non-helical residues are located in the six-residue loop connecting the N34 and C28 helices.

The CD results are also completely consistent with the ¹H-¹⁵N correlation (HSQC) NMR spectrum of N_{CCG}-gp41 (data not shown). A ¹H-¹⁵N HSQC correlation spectrum of uniformly ¹⁵N-labeled N_{CCG}-gp41 was recorded at 40° C. at 600 MHz on a Bruker DRX600 NMR spectrometer. The resulting spectrum was reminiscent of that of the complete ectodomain of SIV gp41 (Caffrey et al., J. Mol. Biol., 271:819-826 (1997)) and displays rather limited dispersion of the backbone amide proton resonances (9.3-6.5 ppm), as expected for a predominantly helical protein.

### Example 4

### Biological Activity of N_{CCG}-gp41

A quantitative vaccinia-virus based reporter gene assay (Salzwedel et al., J. Virol., 74:326-333 (2000)) was used to assess the ability of N_{CCG}-gp41 to inhibit HIV-1 Env-mediated cell fusion. N_{CCG}-gp41 was shown to be a potent inhibitor of HIV-1 Env-mediated cell futsion. Fusion between effector cells bearing HIV-1 Env (LAV) on their surface and target cells expressing the chemokine receptor CXCR4 was activated by addition of soluble CD4. The extent of fusion was directly monitored using β-galactosidase (β-gal) activity as a reporter.

Cell Fusion Assay

A modification (Salzwedel et al., J. Virol., 74:326-333 (2000)) of the vaccinia virus-based reporter gene assay employing soluble CD4 was used to determine the effect on HIV Env-mediated cell fusion of N_{CCG}-gp41, and C34 and N36 peptides. NIH-3T3 and B-SC-1 cells (American Type Culture Collection) grown in Dulbecco's modified Eagle's medium supplemented with 10% fetal bovine serum (DMEM 10%), 2 mM L-glutamine, and gentamycin at 50 μg/mL (all from Gibco BRL, Bethesda, Md.), were used for all assays.

Target NIH-3T3 cells were infected with vCBYF1-fusin (Feng et al., Science, 272:872-877 (1996)) to express the CD4 co-receptor, CXCR4, and with vCB21R-LacZ to express the E. coli lacZ gene under the control of the T7 promoter. Infections were done at a multiplicity of infection of ten.

Effector B-SC-1 cells were infected with vCB41 (Broder et al., Proc. Natl. Acad. Sci. U.S.A., 92:9004-9008 (1995)) to express the HIV-I (LAV) Env protein and with vP11T7genel (Alexander et al., J. Virol., 66:2934-2942 (1992)) to express bacteriophage T7 RNA polymerase. Infections were done at a multiplicity of infection of 10.

In the presence of soluble CD4 protein, fusion of the target and effector cells is mediated by HIV-I Env through the activity of gp120 and gp41. After fusion occurs, bacteriophage T7 RNA polymerase from the effector cell activates transcription of the E. coli lacZ gene contributed by the target cell. Activity of the product of the lacZ gene, β-galactosidase, serves as a quantifiable marker of cell fusion.

Inhibitory activity of N_{CCG}-gp41 was compared to C34 and C36. (Lu et al., Nature Struct. Biol., 2:1075-1082 (1995)) C34 (SEQ ID NO:19) corresponds to the C28 portion of N_{CCG}-gp41 plus an additional six residues at its-C-terminus; N36 corresponds to the N35 portion of N_{CCG}-gp41 plus an additional residue at its C-terminus. N36 (residues 546-581 of HIV-1 Env) and C34 (residues 628-661 of HIV-1 Env) peptides, acetylated at their N-termini and amidated at their C-termini, were synthesized by solid-phase peptide synthesis (Commonwealth Biotechnologies, Richmond, Va.), purified by reverse phase HPLC and characterized by mass spectrometry.

Infected NIH-3T3 and B-SC-1 cells were maintained overnight at 32° C. to allow vaccinia virus mediated expression of the recombinant proteins. The following day, cells were washed, suspended in medium, and used for fusion assays. Assays were carried out in 96 well plates. N_{CCG}-gp41, C34 or N36 were added to an appropriate volume of DMEM 2.5% and PBS to yield identical buffer compositions. 1×10⁵ effector cells in 50 μL media were then added to each well. After incubation for 15 minutes, 1×10⁵ target cells, also in 50 μL media, and soluble CD4 were added to each well. Two-domain soluble CD4 (1-183) was a gift from E. Berger (NIAID, NIH) and donated by S. Johnson (Pharmacia Upjohn, Kalamazoo, Mich.). The final concentration of soluble CD4 was 200 nM. A-fter incubation for 2.5 hours, β-galactosidase activity of cell lysates was measured (A₅₇₀, Molecular Devices 96-well spectrophotometer) using chlorophenol red-β-D-galactopyranoside (Roche, Nutley, N.J.) as a substrate. At least two independent experiments (performed in duplicate) were conducted for each inhibitor. The curves for % fusion versus inhibitor concentration, [I], were fit by non-linear optimization to the activity relationship: % fusion=100/(1+[I]/IC₅₀).

Inhibition of HIV-1 Env-mediated Cell Fusion by N_{CCG}-gp41

Fusion activity as a function of N_{CCG}-gp41 concentration is shown in FIG. 4. N_{CCG}-gp41 inhibits HIV-1 Env-mediated cell fusion at nanomolar concentrations with an IC₅₀ of 16.1±2.8 nM. Parallel experiments were done with the C34 and N36 peptides (Lu et al., Nature Struct. Biol., 2:1075-1082 (1995)) derived from the N- and C-terminal helices of gp41.

The C34 peptide has an IC₅₀ of 2.2±0.5 nM, in agreement with previous studies (Chan et al., Proc. Natl Acad. Sci. U.S.A., 95:15613-15617 (1998); Ferrer et al., Nature Struct. Biol., 6:953-960(1999)). (For comparison, DP-178, the original C-peptide shown to have fusion inhibitory activity (Wild et al., Proc. Natl. Acad. Sci. U.S.A., 91:9770-9774 (1994)) and comprising residues 638-673 of HIV-1 Env which overlaps with the C-terminal half of the C34 peptide, has an IC₅₀ of ˜50 nM (Ferrer et al., Nature Struct. Biol., 6:953-960 (1999)). The fusion inhibitory activity of the N36 peptide, however, is much lower, in the micromolar range with an IC₅₀ of 16.4±1.8 μM, also consistent with previous work (Wild et al., Proc. Natl. Acad. Sci. U.S.A., 89:10537-10541 (1992)).

Fully inhibitory concentrations of N_{CCG}-gp41, C34, or N36, in the presence or absence of CD4, were added to effector cells. The effector cells were then washed repeatedly prior to adding the effector cells to target cells in the presence of soluble CD4. Under these conditions, cell fusion is observed in all three cases, suggesting that all three molecules act on a fusion intermediate of gp41 generated subsequent to the interaction of HIV-1 envelope with cellular receptors, in agreement with previous studies on peptides derived from the C-helix of gp41 (Furuta et al., Nature Struct. Biol., 5:276-279 (1998)).

Increasing the Solubility of N_{CCG}-gp41 at Neutral pH.

The charged surface residues of the C28 domain are identified in Table 5. Eight charged residues are identified. The charged residues are systematically altered in various combinations to neutral polar residues, e.g. Ser, S; Asn, N; Gln, Q; or Thr, T. Alternatively, various combinations of charged residues are changed to oppositely charged residues, e.g. Glu to Asp, Lys to Arg, and vice versa. After the charged residues are altered, functional and biophysical characteristics of the protein are analyzed as described above, as well as the solubility of the protein at neutral pH.

### EXAMPLE 5

### Chemical Synthesis and Cloning of N35_{CCG}-N13 and N34_{CCG}

Design of N34_{CCG} and N35_{CCG}-N13 constructs—In the pre-hairpin intermediate state, formed subsequent to the interaction of gp120 with CD4 and the chemokine coreceptor, the trimeric coiled-coil of N-helices as the C-terminal region of the gp41 ectodomain are exposed. The pre-hairpin intermediate subsequently collapses to form a trimer of hairpins (whose structure has been solved by NMR and crystallographically) bringing the target and viral membranes into aposition. There are three classes of inhibitors that target the pre-hairpin intermediate and prevent its collapse into the trimer of hairpins, thereby rendering it fusion incompetent. (FIG. 1.) Class 1 (e.g. C34) target the exposed trimeric coiled-coil of N-helices; class 2 (e.g. N_{CCG}-gp41, N34_{CCG} and N35_{CCG}-N13) target the C-region; and class 3 (e.g. N₃₆^{Mut(e,g)} interact with the pre-hairpin state to form heterotrimers.

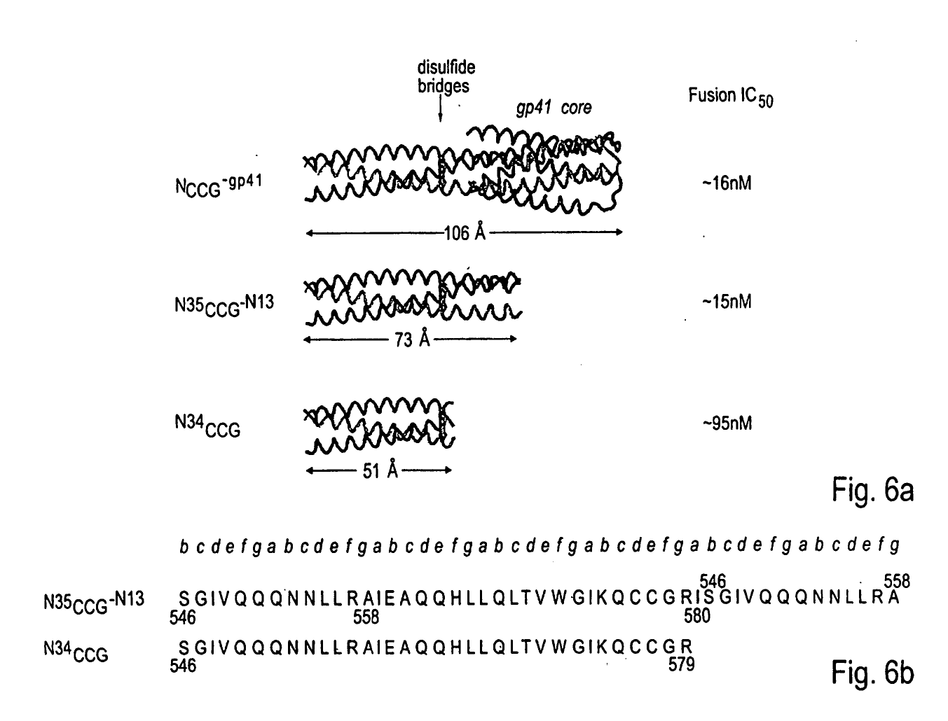

The N34-(L6)-C28 gp41 core has been solved crystallographically. Models of N_{CCG}-gp41, N35_{CCG}-N13, and N34_{CCG} are shown in FIG. 6a. The model of N_{CCG}-gp41 was constructed by grafting N35 onto the N-terminus of the crystal structure to generate a contiguous 69 residue helix comprising N35 and N34. The models of N35_{CCG}-N13 and N34_{CCG} are directly derived from N_{CCG}-gp41. Sequences of N35_{CCG}-N13, N34_{CCG} and N36^{Mut(e,g)} are shown in FIG. 6b. The residue numbering is that of HIV-1 Env. Engineered Cys-Cys-Gly at positions 576-578 have replaced the wild type Leu-Gln-Ala sequence. N13 (residues 546-558 of HIV-1 env) is present twice in N35_{CCG}-N13: once as part of the exposed N-terminal trimeric coiled-coil. The mutations in the native sequence of N36 that were introduced into N36^{Mut(e,g)} are shown. The letters a-g indicate the positions in a helical wheel presentation.

Peptides—The C34 (residues 628-661 of HIV-1 env) and N36^{Mut(e,g)} (residues 546-581 of HIV-1 env with V549D, A551E, L556T, A558L, Q5631, L565E, V570Q, G572K, Q577L mutations) peptides, purchased from Commonwealth Biotechnologies (Richmond, Va.), were synthesized on a solid phase support, purified by reverse phase high pressure liquid chromatography (HPLC), and verified for purity by mass spectrometry and amino acid composition. (Bewley et al., J. Biol. Chem. 277:14238-14245 (2002)). The two peptides bear an acetyl group at the N-terminus and an amide group at the C-terminus.

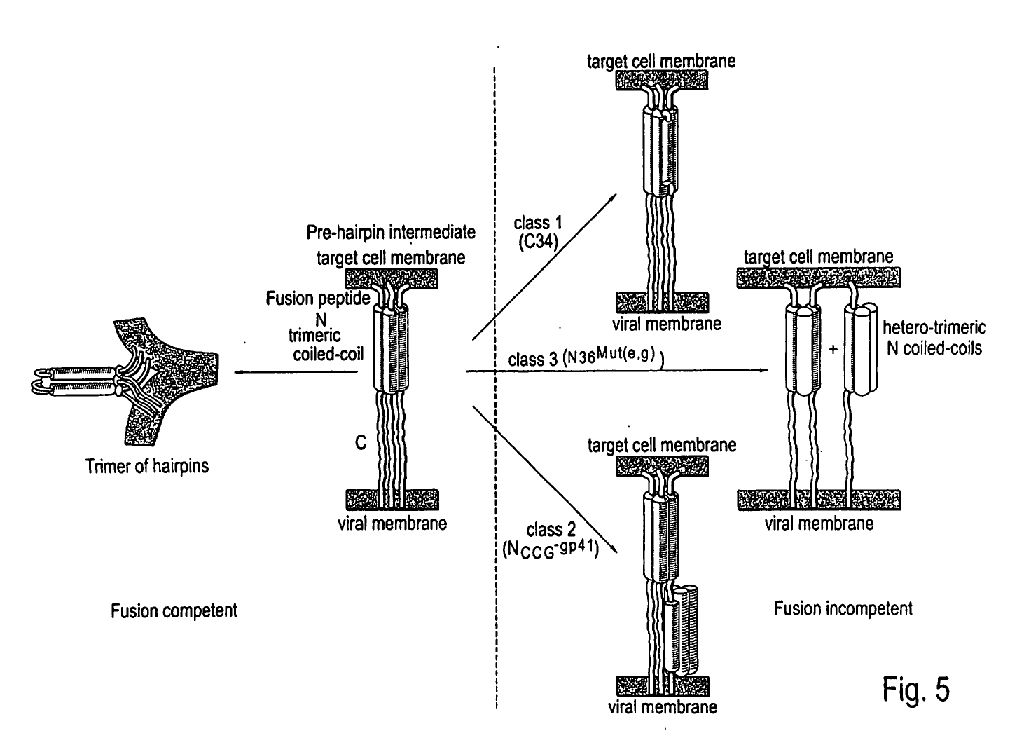

Generation of N34_{CCG} and N35_{CCG}-N13 constructs—The synthesis and cloning of the chimeric protein N_{CCG}-gp41 (FIGS. 1 and 5) has been described previously (Louis et al., J Biol Chem. 276:29485-9 (2001)). N_{CCG}-gp41 comprises N35_{CCG}-N34-(L6)-C28, where N35_{CCG} is residues 546-580 of HIV-1 env with L576C, Q577C and A578G mutations, N34 is residues 546-579 of HIV-1 env, (L6) is a six residue SGGRGG linker, and C28 is residues 628-655 of HIV-1 env. The insert spanning the N35_{CCG}-N34-(L6)-C28 domains was isolated by restriction digestion with NdeI and BamH1 enzymes, and cloned into the pET15b vector (Novagen, Madison, Wis.). The resulting construct 6H-N_{CCG}-gp41, which has a 21 residue 6-His tag at its N-terminus (MGSSHHHHHHSSGLVPRGSHM), was subsequently used to generate the N34_{CCG} and N35_{CCG}-N13 constructs using the purified primers 5′-CTCACGGTCTGGGGCATCAAACAATGTTGTGGCCGCTAGTCCGGCATTGTGCAACAGCAA AACAACTTACTCGC and 5′-GTGCAACAGCAAAACAACTTACTGCGCGCGTAAGAAGCGCAGCAGCACCTGITACAGTT G and their complements, respectively, together with the Quick-Change mutagenesis protocol (Stratagene, La Jolla, Calif.). To construct the minimal trimeric core of gp41, 6H-N34-(L6)-C28, containing a 6-His Tag at its N-terminus, a Ndel site was first created by changing the DNA sequence CGCATC (the six nucleotides upstream to N34) into CATATG using the primer 5′-ACGGTCTGGGGCATCAAACAACTGCAAGCGCATATGTCCGGCATTGTGCAACAG CAAAACAACTTACTGCGCGCG and its complement, the 6H-N_{CCG}-gp41 template and the same mutagenesis' protocol as above. The resulting intermediary construct was digested with Ndel and BamH1 enzymes, and the DNA fragment encoding the N34-(L6)-C28 domains was purified and cloned into pET15b vector. All constructs were expressed in Escherichia coli BL21(DE3). The composition of all expressed proteins was verified by mass spectrometry.

### Example 6

### Expression of N35_{CCG}-N13 and N34_{CCG}

Purification and Protein Folding—Cells were grown at 37° C. in Luria-Bertani medium, induced with 2 mM isopropyl-β-D-thiogalactoside for 4 h, and harvested. The trimeric, intermolecular disulfide-linked, form of N_{CCG}-gp41 was prepared exactly as described previously (Louis et al., J Biol Chem. 276:29485-9 (2001)). To produce the trimeric, intermolecular disulfide-linked, forms of the N34_{CCG} and N35_{CCG}-N13, 2 gm of cells were suspended in 40 ml of 6M guanidine hydrochloride (GnHCL), 50 mM Tris-HCl, pH 8.0, 1 mM 2-mercaptoethanol (buffer A) and lysed by sonication, followed by centrigugation at 16,000 rpm (SS-34 rotor; Sorvall, Newtown, Conn.) for 30 min at 18° C. The supernatant was subjected to Ni-NTA-agarose affinity column (10 ml bed volume) chromatography at room temperature. The column was washed in buffer A and bound protein was eluted in the same buffer containing 0.2 M imidazole. The protein was concentrated on a centriprep YM-3 device (Millipore Corporation, Bedford, Mass.) and applied at room temperature at a flow-rate of 3 ml/min to a Superdex-75 column (HiLoad, 2.6×60-cm; Amersham Biosciences, Piscataway, N.J.) equilibrated in 50 mM Tris-HCl, pH 8, 4 M GnHCL, 5 mM EDTA, and 5 mM dithiothreitol. Peak fractions were then subjected to reverse-phase HPLC on POROS 20 R2 resin (Perceptive Biosystems, Framingham, Mass.) using a linear gradient of 0 to 60% acetonitrile/0.05% trifluoroacetic acid. Peak fractions were pooled and stored at −80° C. Approximately 2.2 mg of either N34_{CCG} or N35_{CCG}-N13 protein in 35% acetonitrile/0.05% TFA/H₂O at a concentration of 0.3 mg/ml was added to 3.2 mg of C34 peptide (residues 628-661 of HIV-1-env). The polypeptide mixture (N34_{CCG}+C34 or N35_{CCG}-N13+C34), kept in a Slide-A-Lyzer cassette (3.5 MWCO; Pierce Chemical Company, Rockford, Ill.), was folded by dialysis against 2 L of 50 mM sodium formate buffer, pH 3.0, for 15 h at room temperature. The intermolecular disulfide bonds were then allowed to form by oxidation using the following dialysis scheme: 20 mM sodium phosphate, pH 6.25, for 2 h; 50 mM sodium formate, pH 3.0, for 3 h; 20 mM sodium phosphate, pH 4.25, for 15 h; and finally 50 mM sodium formate, pH 3.0, for 24 h. The N34_{CCG}·C34 and N35_{CCG}-N13·C34 complexes were concentrated to ˜1 ml and analyzed by SDS-PAGE under non-reducing conditions to verify that the complexes were predominantly disulfide-linked trimers ((Louis et al., J Biol Chem. 276:29485-9 (2001)). The N34_{CCG}·C34 and N35_{CCG}-N13·C34 complexes were subsequently denatured in 7.5 M GnHCL, applied on a Superdex-75 column, and fractionated under denaturing conditions in 4 M GnHCl, 50 mM sodium formate, pH 4. The peak fractions corresponding to the trimeric, disulfide-linked, N34_{CCG} or N35_{CCG}-N13 proteins, stripped of C34, were pooled, concentrated, and stored at 4° C.

Concentrations of all samples were determined spectrophotometrically: the calculated A₂₈₀ values (1 cm path length) for a concentration of 1 mg/ml of N_{CCG}-gp41, N34_{CCG}, N35_{CCG}-N15, N36^{Mut[e,g]} and C34 are 2.026, 0.987, 0.786, 1.31, and 2.90, respectively. The corresponding molecular masses as monomers are 11863, 6011, 7546, 4293 and 4286 Da, respectively.

### Example 7

### Biophysical Properties of N35_{CCG}-N13 and N34_{CCG}

Circular dichroism-CD spectra of N34_{CCG} (10 μM) and N35_{CCG}-N15 (8 μM) were recorded in 20 mM sodium formate buffer, pH 3.0, at 25° C. on a JASCO J-720 spectropolarimeter using a 0.05 cm path length cell. Quantitative evaluation of secondary structure from the CD spectrum was carried out using the program CDNN (www.bioinformatik.biochemtech.uni-halle.de/cd spect/index.html; Andrade et al., Protein Eng., 6:383-390 (1993)).

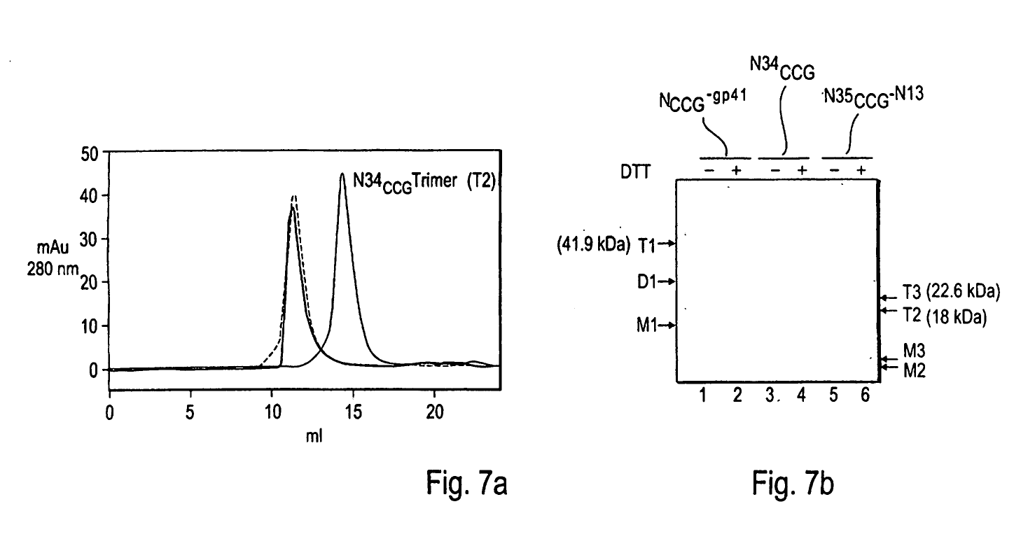

Preparation and characterization of disulfide-linked trimers of the N34_{CCG} and N35_{CCG}-N13 analogs of the internal trimeric coiled-coil of gp41—The chimeric protein N_{CCG}-gp41 folds spontaneously into a trimer which becomes disulfide-linked upon air oxidation. In contrast, the same procedure applied to both 6H-N34_{CCG} and 6H-N35_{CCG}-N13 yields trimers only to an extent of ˜10%. 100% yield of disulfide-linked trimer of 6H-N34_{CCG} and 6H-N35_{CCG}-N13, however, can readily be obtained in a three step procedure: N34_{CCG} and N35_{CCG}-N13 are first folded in the presence of C34 peptide (which comprises the C-helix region of the trimer of hairpins in the fusogenic/post-fusogenic state of gp41). This yields a trimer of the form (N³⁴_{CCG}·C³⁴)₃ (equivalent to the ectodomain core of fusogenic/post-fusogenic gp41) and (N35_{CCG}-N13·C34)₃, as evidenced by the elution profile of the complex at pH 3.0 on a Superdex-75 column (FIG. 7a, profile shown by the dashed line for (N34_{CCG}·C34)₃). This elution pattern is nearly the same as that for N_{CCG}-gp41 which only forms trimers (FIG. 7a, profile shown by the black line). Intermolecular disulfide bond formation between the three chains of N34_{CCG} or N35_{CCG}-N13 in the (N34_{CCG}·C34)₃ or (N35_{CCG}-N13·C³⁴)₃ complexes is then achieved by air oxidation, upon shifting the pH of the solution to 6.25. Finally, the 6H-N34_{CCG} and 6H-N35_{CCG}-N13 disulfide-linked trimers are stripped from the C34 peptide by denaturation in 7.5 M GnHCl followed by size-exclusion column chromatography (in 4 M GnHCl) and reverse-phase HPLC. SDS-polyacrylamide gel analysis of 6H-N34_{CCG} and 6H-N35_{CCG}-N13 subsequent to folding from ˜35% acetonitrile/0.05% TFA by dialysis against 50 mM sodium formate buffer, pH 3, is shown in FIG. 7b. Both 6H-N34_{CCG} and 6H-N35_{CCG}-N13 are completely trimeric under non-reducing conditions (FIG. 7b, lanes 3 and 5, bands labeled T2 and T3, respectively). Treatment of the samples with a reducing agent prior to electrophoresis clearly results in both 6H-N34_{CCG} and 6H-N35_{CCG} migrating to the position of a monomer (FIG. 7b, lanes 4 and 6, labeled M2 and M3, respectively).

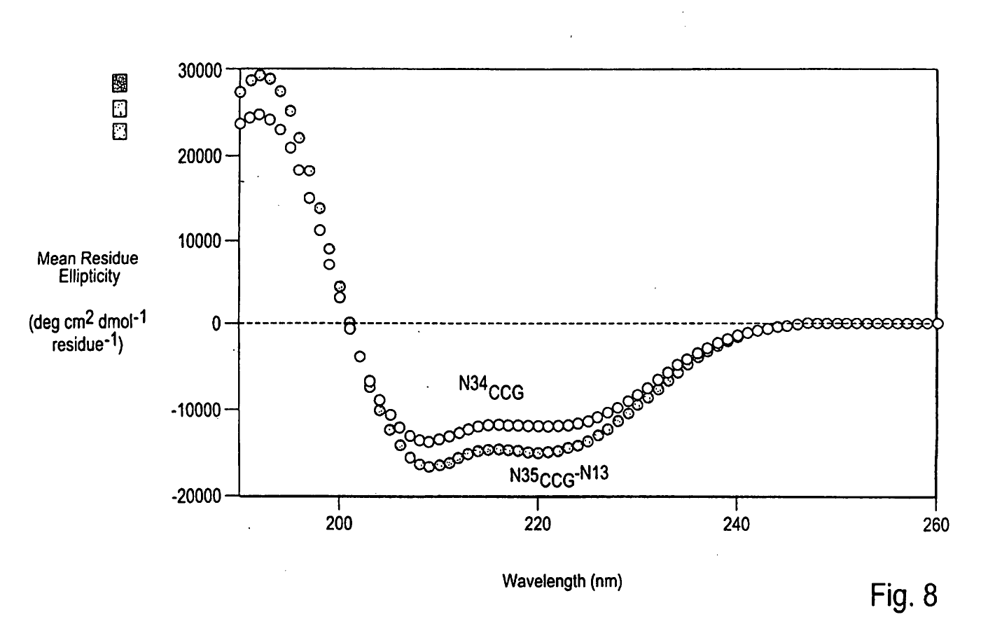

CD spectra of disulfide-linked trimeric N34_{CCG} and N35_{CCT}-N13—CD spectra of disulfide-linked trimeric 6H-N34_{CCG} and 6H-N35_{CCG}-N13 are shown in FIG. 8. Both spectra display a double minimum at 208 and 222 nm, indicative of the presence of α-helix. Quantitative analysis of the spectra using the neural network program CDNN yields an overall helical-content of 43.0±1.5% for 6H-N34_{CCG} and 51.4±0.9% for 6H-N35_{CCG}-N13. These values, however, also reflect the presence of the 21 residue 6His-linker at the N-terminus which is known to be random coil. Thus, the number of helical residues, per subunit, present in trimeric 6H-N34_{CCG} and 6H-N35_{CCG}-N13 is 23.7±0.8 and 35.5±0.6, respectively. The difference in the number of helical residues between N34_{CCG} and N35_{CCG}-N13 reflects the 14 residue extension of the trimeric coiled-coil in N35_{CCG}-N13. Assuming the helical residues are located exclusively within the N34_{CCG} and N35_{CCG}-N13 regions, yields percentage helicities of 69.7±2.4% for N34_{CCG} and 74.0±1.3% for N35_{CCG}-N13.

The CD data is therefore consistent with the models of N34_{CCG} and N35_{CCG}-N13 displayed in FIG. 6a. The models, however, in FIG. 6a are depicted as fully helical. In reality, it is clear from the CD data that the N-terminal 9-10 residues of both constructs are likely to be frayed, and in the case of N35_{CCG}-N13, possibly the last 1-2 C-terminal residues as well.

### Example 8

### Biological Activity of N35_{CCG}-N13 and N34_{CCG}

Cellfusion assay—Inhibition of HIV-Env mediated cell fusion by N34_{CCG}, N35_{CCG}-N13, N_{CCG}-gp41 and the various antibodies was carried out as described previously (Louis et al., J Biol Chem. 276:29485-9 (2001)) using a modification of the vaccinia virus-based reporter gene assay (employing soluble CD4 at a final concentration of 200 nM). B-SC-1 cells were used for both target and effector cell populations. Target cells were co-infected with vCB21R-LacZ and vCBYF1-fusin (CXCR4), and effector cells with vCB41 (Env) and vP11T7gene1, at an MOI of 10. For inhibition studies, proteins or antibodies were added to an appropriate volume of DMEM 2.5% and PBS to yield identical buffer compositions (100 μL), followed by addition of 1×10⁵ effector cells (in 50 μL media) per well. After incubation for 15 min., 1×10⁵ target cells (in 50 μL) and soluble CD4 were added to each well. Following 2.5 hr. incubation, β-galactosidase activity of cell lysates was measured (A₅₇₀, Molecular Devices 96-well spectrophotometer) upon addition of chorophenol red-β-D-galactopyranoside (Roche, Nutley, N.J.).

The curves for % fusion versus peptide inhibitor concentration were fit by non-linear least-squares optimization using the program Kaleidagraph.

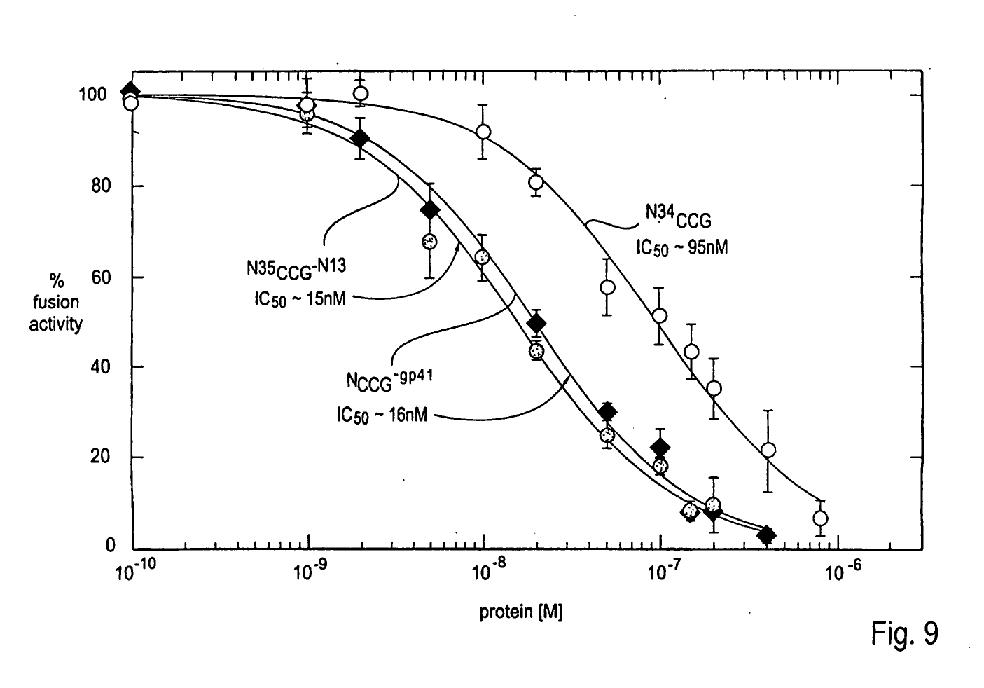

N34_{CCG} and N35_{CCG}-N13 are potent inhibitors of HIV Env-mediated cell fusion—The results of a quantitative vaccinia virus-based reporter gene assay for HIV Env-mediated cell fusion are shown in FIG. 9. The IC₅₀ values for N34_{CCG} and N35_{CCG}-N13 are 96±7 nM and 15.5±1.3 nM. Also shown for comparison in FIG. 5 is the inhibition curve for N_{CCG}-gp41 which has an IC₅₀ 19.3±1.4 nM, consistent with previous data. Thus, one can conclude that N35_{CCG}-N13 is equipotent with N_{CCG}-gp41, and the presence of the additional N13 segment coupled with the intermolecular disulfide bridge is sufficient to stabilize the appropriate region of the trimeric coiled-coil of N-helices in N35_{CCG}-N13. The 5-6 fold lower inhibitory activity of N34_{CCG} relative to both N35_{CCG}-N13 and N_{CCG}-gp41 is presumably due to its slightly lower helical content, as a consequence of fraying at the N-terminus.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A trimeric polypeptide complex consisting of three polypeptide subunits;
a) wherein each subunit comprises between 30 and 50 amino acids from an N-terminal domain of gp41 protein from HIV at the N-terminus of the subunit, with the proviso that the subunit does not include a carboxy-terminal domain of gp41 protein from HIV;
b) wherein the N-terminal domain of the subunit has at least 80% sequence identity to an N34_{CCG} protein of FIG. 6b;
c) wherein the N-terminal domain of the subunit has an amino terminus and a carboxy terminus, and wherein the N-terminal domain of the subunit further has at least two cysteine residues in the ten residues from the carboxy terminus of the domain, and said cysteine residues are able to cross-link with cysteine residues in two other polypeptide subunits of the trimeric polypeptide complex;
d) wherein the N-terminal domain of the subunit forms an exposed trimeric coiled-coil domain having at least 40% alpha helical content when allowed to assemble with two other subunits into a disulfide bridge stabilized trimeric polypeptide complex; and
e) wherein the trimeric polypeptide complex inhibits cell fusion in an HIV based membrane fusion assay.

2. The trimeric polypeptide complex of claim 1, wherein the polypeptide subunits comprise the amino acid sequence of the N34_{CCG} protein of FIG. 6b.

3. The trimeric polypeptide complex of claim 1, wherein the polypeptide subunits further comprise a second N-terminal domain of gp41 attached to the carboxy terminus of the subunit.

4. The trimeric polypeptide complex of claim 3, wherein the polypeptide subunits further coamprise 1-13 residues of an N-terminal domain of gp41.

5. The trimeric polypeptide complex of claim 3, wherein the polypeptide subunits have at least 80% identity to an N35_{CCG}-N13 protein of FIG. 6b.

6. The trimeric polypeptide complex of claim 1, wherein the N-terminal domain of the subunit forms an exposed trimeric coiled-coil domain having at least 50% alpha helical content when allowed to assemble with two other subunits into a disulfide bridge stabilized trimeric polypeptide complex.

7. The trimeric polypeptide complex of claim 1, wherein the polypeptide subunits further comprise a His-tag sequence.

8. The trimeric polypeptide complex of claim 1, wherein the trimeric polypeptide complex is included in a pharmaceutical excipient suitable for administration to a human, in an amount sufficient to generate an immune response.

9. A method of protecting a human from HIV infection by administering to the human an amount of an immunogenic composition comprising:
a trimeric polypeptide complex consisting of three polypeptide subunits;
a) wherein each subunit comprises between 30 and 50 amino acids from an N-terminal domain of gp41 protein from HIV at the N-terminus of the subunit, with the proviso that the subunit does not include a carboxy-terminal domain of gp41 protein from HIV;
b) wherein the N-terminal domain of the subunit has at least 80% sequence identity to an N34_{CCG} protein of FIG. 6b;
c) wherein the N-terminal domain of the subunit has an amino terminus and a carboxy terminus, and wherein the N-terminal domain of the subunit further has at least two cysteine residues in the ten residues from the carboxy terminus of the domain, and said cysteine residues are able to cross-link with cysteine residues in two other polypeptide subunits of the trimeric polypeptide complex;
d) wherein,the N-terminal domain of the subunit forms an exposed trimeric coiled-coil domain having at least 40% alpha helical content when allowed to assemble with two other subunits into a disulfide bridge stabilized trimeric polypeptide complex; and
e) wherein the trimeric polypeptide complex inhibits cell fusion in an HIV based membrane fusion assay;
the amount of immunogenic composition being sufficient to induce an anti-HIV immune response.

10. The method of claim 9, wherein the polypeptide subunits comprise the amino acid sequence of the N34_{CCG} protein of FIG. 6b.

11. The method of claim 9, wherein the polypeptide subunits further comprise a second N-terminal domain of gp41 attached to the carboxy terminus of the subunit.

12. The method of claim 11, wherein the polypeptide subunits further comprise 1-13 residues of an N-terminal domain of gp41.

13. The method of claim 11, wherein the polypeptide subunits have at least 80% identity to an N35_{CCG}-N13 protein of FIG. 6b.

14. The method of claim 9, wherein the N-terminal domain of the subunit forms an exposed trimeric coiled-coil domain having at least 50% alpha helical content when allowed to assemble with two other subunits into a disulfide bridge stabilized trimeric polypeptide complex.

15. The method of claim 9, wherein the polypeptide subunits further comprise a His-tag sequence.

16. An immunogen capable of inducing a response against an exposed trimeric coiled-coil domain from an N-terminal domain of gp41 protein from HIV comprising:
the molecule of claim 1,
wherein said molecule is soluble in an aqueous solution of pH 7 at a concentration of at least 0.5 micromolar.

17. A trimeric polypeptide complex consisting of three polypeptide subunits;
a) wherein each subunit comprises between 30 and 50 amino acids from an N-terminal domtain of gp41 protein from HIV at the N-terminus of the subunit;
b) wherein the N-terminal domain of the subunit has at least 80% sequence identity to an N34_{CCG} protein of FIG. 6b;
c) wherein the N-termninal domain of the subunit has an amino terminus and a carboxy terminus, and wherein the N-terminal domain of the subunit further has at least two cysteine residues in the ten residues from the carboxy terminus of the domain, and said cysteine residues are able to cross-link with cysteine residues in two other polypeptide subunits of the trimeric polypeptide complex;
d) wherein the N-terminal domain of the subunit forms an exposed trimeric coiled-coil domain having at least 40% alpha helical content when allowed to assemble with two other subunits into a disulfide bridge stabilized trimeric polypeptide complex; and
e) wherein the trimeric polypeptide complex inhibits cell fusion in an HIV based membrane futsion assay.

18. The trimeric polypeptide complex of claim 17, wherein the polypeptide subunits comprise the amino acid sequence of the N34_{CCG} protein of FIG. 6b.

19. The trimeric polypeptide complex of claim 17, wherein the polypeptide subunits further comprise a second N-terminal domain of gp41 attached to the carboxy terminus of the subunit.

20. The trimeric polypeptide complex of claim 17, wherein the polypeptide subunits further comprise 1-13 residues an N-terminal domain of gp41.

21. The trimeric polypeptide complex of claim 17, wherein the polypeptide subunits have at least 80% identity to N35_{CCG}-N13 protein of FIG. 6b.

22. The trimeric polypeptide complex of claim 17, wherein the N-terminal domain of the subunit forms an exposed trimeric coiled-coil domain having at least 50% alpha helical content when allowed to assemble with two other subunits into a disulfide bridge stabilized trimeric polypeptide complex.

23. The trimeric polypeptide complex of claim 17, wherein the polypeptide subunits further comprise a His-tag sequence.

24. The trimeric polypeptide complex of claim 17, wherein the trimeric polypeptide complex is included in a pharmaceutical excipient suitable for administration to a human, in an amount sufficient to generate an immune response.

25. The trimeric polypeptide complex of claim 17, wherein the carboxy terminus of the N-terminal domain of the subunit is fused to an amino terminus of a six helix bundle domain, and further,
has at least 90% alpha helical content when the N-terminal domain of the subunit is fused to a six helix bundle domain and allowed to assemble with two other subunits into a disulfide bridge stabilized trimeric protein.

26. The trimeric polypeptide complex of claim 25, wherein the N-terminal domain of the subunit forms a trimeric protein having 90% alpha helical content when the N-terminal domain of the subunit is fused to the six helix bundle domain of SEQ ID NO:4 and allowed to assemble with two other subunits into a disulfide bridge stabilized trimeric protein

27. The trimeric polypeptide complex of claim 25, wherein the six helix bundle domain is selected from the group consisting of: the gp41 protein of HIV-1, the gp41 protein of SIV, and GCN4.

28. The trimeric polypeptide complex of claim 25, wherein the six helix bundle domain comprises an N34 domain linked to a C28 domain wherein:
i. the N34 domain is has between 30 and 50 amino acid residues having an amino terminus and carboxy terminus of N34;
ii. wherein at least 30 amino acid residues of the total amino acid residues of N34 have more than an 80% sequence identity to SEQ ID:2,
iii. the C28 domain has between 25 and 45 amino acid residues having an amino terminus and carboxy terminus of C28;
iv. wherein at least 28 amino acid residues of the total amino acid residues of C28 have more than an 80% sequence identity to SEQ ID:3; and
v. wherein the rarboxy terminus of the N34 domain is linked to the amino terminus of the C28 domain by a linker of between 4 and 12 amino acids.

29. The trimeric polypeptide complex of claim 17, wherein the N-terminal domain of gp41 protein from HIV comprises SEQ ID NO: 1.

30. The trimeric polypeptide complex of claim 17, wherein the N-terminal domain of gp41 protein from HIV is SEQ ID NO: 1.

31. The protein of claim 28, wherein the C28 domain has at least 80% identity to SEQ ID NO:3.

32. The protein of claim 28, wherein the polypeptide subunits comprise SEQ ID NO:5.

33. The trimeric polypeptide complex of claim 17, wherein the protein is included in a pharmaceutical excipient suitable for administration to a human, in an amount sufficient to generate an immune response.

34. A method of protecting a human from HWV infection by administering to the human an amount of a immunogenic composition comprising:
a trimeric polypeptide complex consisting of three polypeptide subunits;
a) wherein each subunit comprises between 30 and 50 amino acids from an N-terminal domain of gp41 protein from HIV at the N-terminus of the subunit;
b) wherein the N-terminal domain of the subunit has at least 80% sequence identity to an N34_{CCG} protein of FIG. 6b;
c) wherein the N-terminal domain of the subunit has an amino terminus and a carboxy terminus, and wherein the N-terminal domain of the subunit further has at least two cysteine residues in the ten residues from the carboxy terminus of the domain, and said cysteine residues are able to cross-link with cysteine residues in two-other polypeptide subunits of the trimeric polypeptide complex;
d) wherein the N-terminal domain of the subunit forms an exposed trimeric coiled-coil domain having at least 40% alpha helical content when allowed to assemble with two other subunits into a disulfide bridge stabilized trimeric polypeptide complex; and
e) wherein the trimeric polypeptide complex inhibits cell fusion in an HIV based membrane fusion assay;
the amount of immunogenic composition being sufficient to induce an anti-HIV immune resppnse.

35. The trimeric polypeptide complex of claim 34, wherein the polypeptide subunits comprise the amino acid sequence of the N34_{CCG} protein of FIG. 6b.

36. The trimeric polypeptide complex of claim 34, wherein the polypeptide subunits further comprise a second N-terminal domain of gp41 attached to the carboxy terminus.

37. The trimeric polypeptide complex of claim 34, wherein the polypeptide subunits further comprise 1-13 residues an N-terminal domain of gp41.

38. The trimeric polypeptide complex of claim 34, wherein the polypeptide subunits have at least 80% identity to N35_{CCG}-N13 protein of FIG. 6b.

39. The trimeric polypeptide complex of claim 34, wherein the N-terminal domain of the subunit forms an exposed trimeric coiled-coil domain having at least 50% alpha helical content when allowed to assemble with two other subunits into a disulfide bridge stabilized trimeric polypeptide complex.

40. The trimeric polypeptide complex of claim 34, wherein the polypeptide subunits further comprise a His-tag sequence.

41. The trimeric polypeptide complex of claim 34, wherein the trimeric polypeptide complex is included in a pharmaceutical excipient suitable for administration to a human, in an amount sufficient to generate an immune response.

42. The trimeric polypeptide complex of claim 34, wherein the carboxy terminus of the N-terminal domain of the subunit is fused to an amino terminus of a six helix bundle domain, and further,
has at least 90% alpha helical content when the N-terminal domain of the subunit is fused to a six helix bundle domain and allowed to assemble with two other subunits into a disulfide bridge stabilized trimeric protein.

43. The trimeric polypeptide complex of claim 42, wherein the N-terminal domain of the subunit forms a trimeric protein having at least 90% alpha helical content when the N-terminal domain of the subunit is fused to the six helix bundle domain of SEQ ID NO:4 and allowed to assemble with two other subunits into a disulfide bridge stabilized trimeric protein

44. The trimeric polypeptide complex of claim 42, wherein the six helix bundle domain is selected from the group consisting of: the gp41 protein of HIV-1, the gp41 protein of SIV, and GCN4.

45. The trimeric polypeptide complex of claim 42, wherein the six helix bundle domain comprises an N34 domain linked to a C28 domain wherein:
i. the N34 domain is has between 30 and 50 amino acid residues having an amino terminus and carboxy terminus of N34,
ii. wherein at least 30 amino acid residues of the total amino acid residues of N34 have more than an 80% sequence identity to SEQ ID:2,
iii. the C28 domain has between 25 and 45 amino acid residues having an amino terminus and carboxy terminus of C28;
iv. wherein at least 28 amino acid residues of the total amino acid residues of C28 have more than an 80% sequence identity to SEQ ID:3; and
v. wherein the carboxy terminus of the N34 domain is linked to the amino terminus of the C28 domain by a linker of between 4 and 12 amino acids.

46. The trimeric polypeptide complex of claim 34, wherein the N-terminal domain of the subunit comprises SEQ ID NO: 1.

47. The trimeric polypeptide complex of claim 34, wherein the N-terminal domain of the subunit is SEQ ID NO: 1.

48. The protein of claim 45, wherein the C28 domain has at least 80% identity to SEQ ID NO:3.

49. The protein of claim 45, wherein the polypeptide subunits comprise SEQ ID NO:5.

50. The method of claim 34, wherein the composition is administered parenterally.

51. An immunogen capable of inducing a response against an exposed trimeric coiled-coil domain from an N-terminal domain of gp41 protein from HIV comprising:
the molecule of claim 17,
wherein said molecule is soluble in an aqueous solution of pH 7 at a concentration of at least 0.5 micromolar.
